(19) United States
(12) Patent Application Publication
(10) Pub. No.: US 2023/0270892 A1
(43) Pub. Date: Aug. 31, 2023

(54) CHELATOR COMPOSITIONS FOR RADIOMETALS AND METHODS OF USING SAME

(71) Applicants: TRIUMF INC., Vancouver (CA); Provincial Health Services Authority, Vancouver (CA); The University of British Columbia, Vancouver (CA)

(72) Inventors: Hua YANG, Vancouver (CA); Feng GAO, Vancouver (CA); Paul SCHAFFER, Vancouver (CA); Zheliang YUAN, Vancouver (CA); Chengcheng ZHANG, Vancouver (CA); Francois BENARD, Vancouver (CA); Luke WHARTON, Vancouver (CA)

(21) Appl. No.: 17/801,777

(22) PCT Filed: Feb. 25, 2021
(86) PCT No.: PCT/CA2021/050226

§ 371 Date: Aug. 23, 2022

Related U.S. Application Data
(60) Provisional application No. 62/993,636, filed on Mar. 23, 2020.
(60) Provisional application No. 62/981,113, filed on Feb. 25, 2020.

(51) Int. Cl. A61K 51/04; C07D 273/00; A61K 51/08

(52) U.S. Cl. CPC A61K 51/044; A61K 51/088; C07D 273/00

(57) ABSTRACT

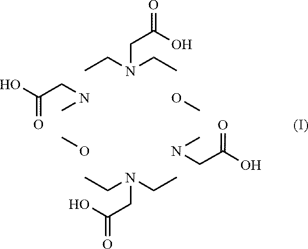

A chelator having the general structure (I) for chelating ra-diometals such as ²²⁵Ac under mild conditions is provided. (I) The chelator can be coupled to a biological targeting moiety to facilitate targeted delivery of the chelated radiometal in a mammalian subject.

### REFERENCE TO RELATED APPLICATIONS

This application claims priority to, and the benefit of, U.S. Provisional Pat. Applications No. 62/981113 filed 25 Feb. 2020 and No. 62/993636 filed 23 Mar. 2020. Both of the foregoing applications are incorporated by reference herein in their entireties.

### TECHNICAL FIELD

Some embodiments relate to improved chelators. Some embodiments relate to improved biological targeting constructs incorporating chelators. Some embodiments relate to chelators coupled to a targeting moiety and capable of binding a radioactive isotope to provide targeted in vivo delivery of the radioactive isotope to a desired location within a mammalian subject.

### BACKGROUND

Radionuclides have potential utility in cancer diagnosis and therapy, particularly if they can be delivered selectively to a target location within the body of a subject. Targeted delivery of radionuclides can be achieved by using constructs that are engineered to both securely retain the radionuclide for in vivo delivery and deliver the radionuclide selectively to a desired location within the body, with a reasonably low level of delivery to non-target regions of the body.

Targeting constructs have been developed that utilize a targeting moiety that targets a desired region of the body (e.g. a tumor-associated antigen) covalently coupled to a chelator to secure radionuclides for such purposes. The targeting moiety can be coupled to the chelator via a linker. Such targeting constructs may be referred to as radioimmunoconjugates. The radioimmunoconjugate is used to chelate a desired radionuclide for in vivo delivery, for example to provide diagnostic imaging, targeted radionuclide therapy using the construct, or both (i.e. as a theranostic construct).

Chelators useful in such constructs may have characteristics such as rapid complexation kinetics and strong affinity for the radionuclide under mild conditions (e.g. low temperature such as room temperature, with complexation to a high degree occurring within the span of several minutes), as well as high versatility of linker incorporation (i.e. bifunctionalization) without sacrificing the coordination integrity. While small peptidomimetics and other such constructs provide targeting moieties that may have higher tolerance for harsher radiolabeling conditions (e.g. at higher temperature), other targeting moieties such as biologics, e.g. antibodies and antigen-binding fragments thereof, may not be tolerant of harsh radiolabeling conditions such as increased temperature (e.g. may not accommodate high labelling temperatures in the range of 60° C. to 90° C. or higher).

Targeted radionuclide therapy (TRT) has been gaining popularity for the treatment of certain cancers, demonstrating significant therapeutic efficacy and survival benefit, especially for later stage disease with limited conventional therapy options.^{[1,2]} Most isotopes used for clinical TRT are beta emitters, including lutetium-177,^{[3]} yttrium-90,^{[4]} strontium-89^{[5]} and samarium-153^{[6]}, with radium-223^{[7]} being the only FDA approved alpha-emitter to date. Alpha emitters have a much higher linear energy transfer (LET, energy deposition per unit pathlength) of ~100 keV/µm compared to beta emitters (1-2 keV/µm), contributing to substantially more free radical (ROS) generation and lethal DNA double-strand breaks.^{[8],[9]} The short range of alpha particles (40-100 µm) can potentially spare surrounding healthy tissues when delivered with tumor-specific targeting vectors, a feature particularly desirable when treating micrometastases. The cytotoxicity of alpha emitters is also independent from cell cycle or oxygenation status.^{[10,11]}

Actinium-225 (²²⁵Ac) is an emerging alpha emitter for targeted alpha therapy (TAT), with its favorable half-life (9.9 days) allowing adequate time for radiopharmaceutical preparation, global isotope distribution, patient administration and blood circulation for longer-resident targeting vectors such as antibodies (5-6 days for IgA and IgM). ²²⁵Ac emits four high-energy alpha particles through a rapid decay chain that contributes to its high cytotoxicity (FIG. 1). This potential is demonstrated by clinical studies on ²²⁵Ac-PSMA-617 with metastatic castration-resistant prostate cancer (mCRPC) in patients that had exhausted all other conventional treatments. Early salvage treatment in two patients showed complete remission.^{[12]} In a larger study in 40 patients, among the 38 patients surviving at least 8 weeks, 24 (63%) had a PSA decline of more than 50%, and 33 (87%) had a PSA response of any degree.^{[13]} Although still experimental, ²²⁵Ac targeted alpha-therapy shows strong potential for providing last-line clinical treatments for other late stage cancer patients. However the availability of chelators for actinium remains limited to a small number of examples.

Research on ²²⁵Ac radiopharmaceuticals is hampered by the limited global isotope supply, and consequently limited chemistry and radiochemistry development to date.^{[9,14]} The DOTA chelator having the structure shown below in Chart 1 is the current workhorse for attaching ²²⁵Ac to peptides, antibodies and other targeting molecules. In fact, it is used in all clinical trials to date. However, DOTA is well known to have decreased thermodynamic stability towards larger metal ions,^{[15]} and chelation to ²²⁵Ac is established to be kinetically slow, making it susceptible to small metal impurities (such as Ca²⁺), requiring extensive heating and high ligand concentration for adequate radiolabeling yields.^{[16-18]} Macropa having the structure shown below in Chart 1 is one example of a promising chelator that can quantitatively label Ac at submicromolar (< 10⁻⁶ M) levels.^{[19,20]} It has recently been used as part of a novel PSMA-targeting radiopharmaceutical (RPS-074), although the bifunctional isothiocyanate derivative is difficult to synthesize and unstable.^{[19]} Several other chelators (bispa,^{[21]} CHXoctapa,^{[22]} and DOTP^{[23]}) demonstrate high Ac binding affinity as well, though in vivo stability has not yet been evaluated. Some other chelators have limited success either due to binding affinity (TETA, TETPA, DOTPA) or in vivo stability (DOTMP, HEHA).^{[9,24]} The intrinsic characteristics of a chelator such as lipophilicity and charge can have profound yet unpredictable impact on the biological function of a radiopharmaceutical,^{[25,26]} and it is important to provide a chelator having favourable properties to be able to fully realize the potential applications ²²⁵Ac holds.

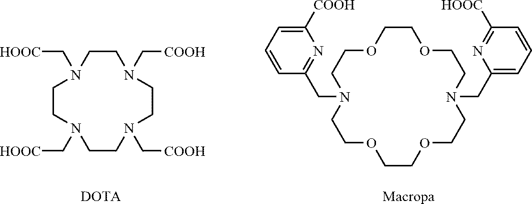

Chart 1. Currently available chelators for ²²⁵Ac.

A chelating agent that can both chelate a metal and be conjugated to a targeting moiety is necessary to bind, with a good stability, a radionuclide to a targeting vector. Currently, chelation strategies for actinium are limited, hindering its clinical application. Chelators for Actinium-225 that can coordinate under mild conditions and produce a stable complex in vivo are needed. Furthermore, good chelators that bind with a high degree of specificity and binding affinity are needed, particularly where targeted alpha-therapy is desired to be applied against receptors or targets that are expressed at low densities and that are therefore readily saturable.

Actinium-225 is potentially useful to conduct targeted alpha-therapy when it can be conjugated to a suitable targeting moiety via a chelator. An example of one targeting moiety that can be used to conduct targeted alpha-therapy with ²²⁵Ac is an α-melanocyte-stimulating hormone (αMSH) derivative, CCZ01048, designed for MC1R-targeted melanoma imaging and treatment, which is a candidate that has been shown to exhibit rapid tumor uptake and internalization and was chosen for subsequent functionalization and in vivo uptake studies in tumor bearing mice.^{[29]} Late stage metastatic melanoma is a deadly disease with low long-term survival rate even with immunotherapy agents.^{[30-33]} There is currently no curative option available for this disease. MC1R is specifically expressed in primary and metastatic melanoma with low normal tissue expression.^{[34,35]} The inventors have previously developed αMSH-based radiopharmaceuticals targeting MC1R with [68Ga]Ga-CCZ01048^{[29]} and [18F]CCZ01064^{[36]} for positron emission tomography (PET) imaging in a preclinical model of mouse B16F10 melanoma. The inventors have also evaluated a novel αMSH-based [18F]CCZ01096 radiotracer in a preclinical model of human melanoma with the SK-MEL-1 cell line.^{[37]} In all three cases, great tumor visualization in PET images was achieved with excellent tumor-to-normal tissue ratios, i.e. average tumor-to-blood and tumor-to-muscle tumor-to-liver ratios were >30 and >90 respectively. The inventors also previously evaluated ²²⁵Ac-CCZ01048 (DOTA).^{[22]} See also PCT application publication No. WO 2019/222851.

Ideally, Ac-radiopharmaceuticals should have low normal tissue uptake and fast tumor internalization to help mitigate any cytotoxicity induced by irradiation of healthy tissue, and ensure alpha-emitting daughter radionuclides released from the targeting vector are contained inside the tumors. An effective chelator that does not release the bound radiometal readily under physiological conditions is important to achieving this.

The structure and synthesis of 2,2’,2’’,2’’’-(1,10-dioxa-4,7,13,16-tetraazacyclooctadecane-4,7,13,16-tetrayl)tetraacetic acid is described in CN 102212042, CN 10415367 and CN 104151368.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In some aspects, an in vivo radioisotope targeting construct has a biological targeting moiety and a chelator having the structure (I), (II), (III), (IV), (V), (VI), (VII), (VIII) or (IX):

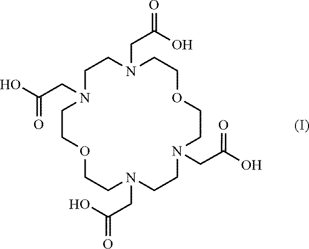

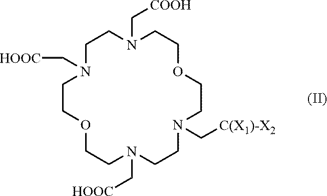

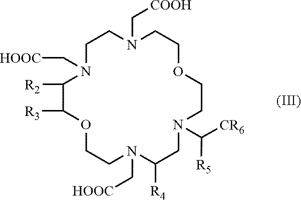

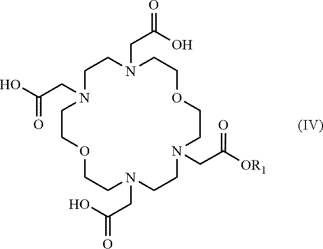

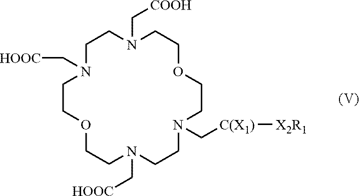

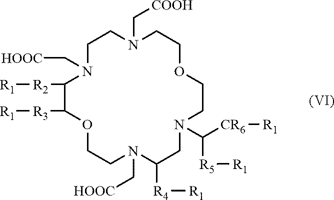

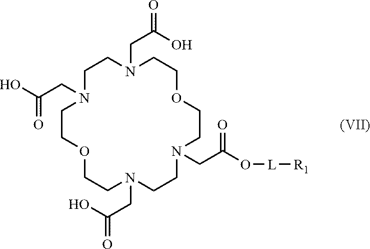

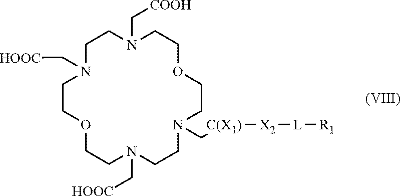

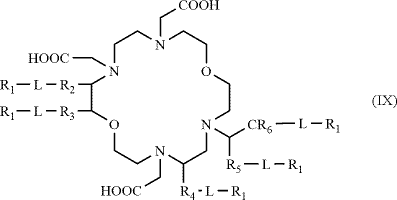

wherein: X₁ and X₂ are independently O, N or S; R₂, R₃, R₄, R₅ and R₆ are independently not present or a functional group that can be used to couple the chelator to a biological targeting moiety, and optionally only one of R₂, R₃, R₄, R₅ and R₆ is present; R₁ when present represents the biological targeting moiety; and L when present represents a linker.

R₂, R₃, R₄, R₅ or R₆ when present can be independently a carboxyl, an ester, an amide, an imide, a thioamide, a thioester, a guanidinium, an ether, a thioether, or an amine group. The linker L when present can be a C₁-C₁₀ hydrocarbon linker that is optionally substituted with one or more heteroatoms or has one or more substituents, an aromatic linker, a cationic linker, an anionic linker, an amino acid linker having between one and ten amino acids, a cyclized amino acid linker, a PEG linker, a cyclized ring linker, an aromatic linker, or a click chemistry linker. The construct can have a radiometal chelated by the chelator, and the radiometal can be ²²⁵Ac, ²¹³Bi, ⁶⁸Ga, ¹⁵⁵Tb, ¹⁷⁷Lu, ¹¹¹ln, or ¹³⁷Cs. The targeting moiety can be a hapten, an antigen, an aptamer, an affibody, an enzyme, a protein, a peptide, an antibody, an antigen-binding fragment of an antibody, a peptidomimetic, a receptor ligand, a steroid, a hormone, a growth factor, a cytokine, a molecule that recognizes cell surface receptors, a lipid, a lipophilic group, or a carbohydrate. The targeting moiety can target any suitable biological target, for example a tumor associated antigen.

In some aspects, a method of delivering a radioisotope to a selected location within the body of a mammalian subject by administering an in vivo radioisotope targeting construct as described herein bearing the radioisotope to the mammalian subject is provided. The targeting moiety can facilitate accumulation of the construct at the selected target location within the body relative to other locations in the body to selectively deliver radiation to the selected location. In some aspects, the localized radioisotope is used to carry out an imaging procedure, e.g. PET or SPECT imaging. In some aspects, the localized radioisotope is used to cause cell death at the selected location by exposing the cells to radiation from the radioisotope. In some aspects, the radiation is alpha radiation. In some aspects, the cells that are killed by the radiation are cancer cells. The mammalian subject may be a human.

In some aspects, a chelate can be formed from the in vivo radioisotope targeting construct and the radioisotope by combining the two together under mild conditions, e.g. at a temperature between about 10° C. to about 65° C. for a period of between about 5 and about 30 minutes at a pH in the range of about 5.0 to about 7.4.

In some aspects, a metal chelate comprising a chelator having the structure (I), (II) or (III) shown above and one of ²²⁵Ac, ²¹³Bi, ⁶⁸Ga, ¹⁵⁵Tb, ¹⁷⁷Lu, ¹¹¹ln, or ¹³⁷Cs is provided. In some aspects, a method of forming a metal chelate by combining a chelator having the structure (I), (II), (III), (IV), (V), (VI), (VII), (VIII) or (IX) above with a radiometal in an aqueous solution at a temperature of between 15° C. and 25° C. is provided. The metal may be ²²⁵Ac, ²¹³Bi, ⁶⁸Ga, ¹⁵⁵Tb, ¹⁷⁷Lu, ¹¹¹ln, or ¹³⁷Cs. The pH may be in the range of about 5.0 to about 7.4. The combining step may be carried out for a period of between about 5 and about 30 minutes. The metal chelate may be stable in and present in mammalian serum or mammalian blood, optionally human serum or human blood. The metal chelate may be present in a mammal, optionally in a human.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 shows the decay chain for ²²⁵Ac.

FIG. 2 shows an example embodiment of an in vivo radioisotope targeting construct incorporating a crown chelator with a linker interposing the crown and the targeting moiety.

FIG. 3 shows an example embodiment of an in vivo radioisotope targeting construct incorporating crown as a chelator without a linker.

FIG. 4 shows an example embodiment of an in vivo radioisotope targeting construct incorporating bifunctional crown as a chelator with a linker interposing the bifunctional crown and the targeting moiety.

FIG. 5 shows an example embodiment of an in vivo radioisotope targeting construct incorporating bifunctional crown as a chelator without a linker.

FIG. 6A represents a scheme for coupling a crown chelator to peptide targeting moieties according to one example embodiment.

FIG. 6B represents a scheme for coupling a crown chelator to peptide targeting moieties according to another example embodiment.

FIG. 7 shows the radioTLC results for an exemplary crown-octreotate (TATE) construct chelating ²²⁵Ac according to an example embodiment.

FIG. 8 shows the radioTLC results for a corresponding control (free Ac).

FIG. 9 shows the labelling yield with ²²⁵Ac at various concentrations of crown chelator ([C]) at room temperature in one experiment.

FIG. 10 shows the radiolabelling yield with ²²⁵Ac at various concentrations of crown chelator (provided as crown-TATE targeting construct) at room temperature in one experiment, during periods of 30 minute or 60 minute incubation.

FIG. 11 shows labelling of crown with ²²⁵Ac in various buffers at various pH levels.

FIG. 12 shows the serum stability of ²²⁵Ac-crown-TATE at 37° C.

FIG. 13 shows the radiochemical yield over time of ²²⁵Ac-labelled crown.

FIG. 14 shows the comparative labelling of crown and DOTA with ²²⁵Ac at various chelator concentrations ([Ligand]).

FIG. 15 shows the serum stability of ²²⁵Ac-crown-αMSH over time.

FIG. 16 shows the comparative labelling of crown, crown-TATE, crown-αMSH and DOTA with ¹⁷⁷Lu at different chelator concentrations.

FIG. 17 shows the labelling of crown and crown-αMSH with ²¹³Bi.

FIG. 18 shows the labelling of crown and crown-αMSH versus DOTA with ¹⁵⁵Tb.

FIG. 19 shows the labelling of crown-TATE with ⁶⁸Ga.

FIG. 20 shows the serum stability of crown, crown-αMSH and crown-TATE with ¹⁷⁷Lu.

FIG. 21 shows the serum stability of ¹⁵⁵Tb-crown-aMSH at 37° C.

FIG. 22 shows the biodistribution of the ²²⁵Ac-crown-αMSH construct (% ID/g) when prepared the night before (A) and prepared the same day (4 h before) with Sep-Pak purification and 0.1 M L-ascorbate (D). (Two-way ANOVA, multiple comparisons corrected using the Sidak method, *** p < 0.0001, n ≥ 3).

FIG. 23 shows HPLC gamma traces of ²²⁵Ac-crown-αMSH with and without 0.1 M L-ascorbate.

FIG. 24 shows the biological distribution of ²²⁵Ac-crown-TATE in mice with B16F10 tumors, as the % injected dose per gram of tissue (% ID/g).

FIG. 25 shows the biological distribution of ²¹³Bi-crown-TATE in mice with B16F10 tumors, as the % injected dose per gram of tissue (% ID/g).

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

As used herein, the term prophylaxis includes preventing, minimizing the severity of, or preventing a worsening of a condition. As used herein, the terms treat or treatment include reversing or lessening the severity of a condition.

As used herein, the term antibody includes all forms of antibodies including polyclonal antibodies, monoclonal antibodies, chimeric antibodies, humanized antibodies, single chain antibodies, multimeric antibodies, and the like. The term antigen binding fragment of an antibody refers to any portion of an antibody that is capable of binding to an antigen and includes by way of example only and without limitation Fab fragments, F(ab’)₂ fragments, Fv fragments, scFv fragments, minibodies, diabodies, and the like. Reference to a specific antibody includes reference to any antibodies that are determined to be biosimilar to that specific antibody by any regulatory authority.

As used herein, the term peptidomimetic means a small protein-like molecule designed to mimic a peptide, and includes without limitation modified peptides, peptidic foldamers, structural mimetics and mechanistic mimetics.

A chelator composition for radiometals is disclosed. A method of using and making the composition is also disclosed. The composition can be used as a therapeutic and/or diagnostic agent.

The inventors have now determined that chelators having the general structure (1) can coordinate radioisotopes including ²²⁵Ac under mild conditions and produce a complex that is stable under in vivo conditions, making such chelators particularly suitable for example for application in radiotherapeutic, diagnostic and/or theranostic constructs. The chelator can be coupled directly or via a linker to a biological targeting moiety to create a construct suitable for use in such applications.

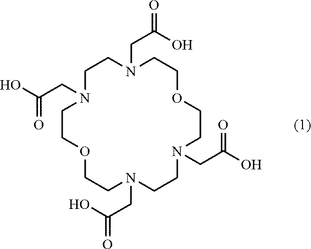 [SMILES: O=C(O)CN1CCOCCN(CC(=O)O)CCN(CC(=O)O)CCOCCN(CC(=O)O)CC1]

The structure (1) represents 2,2’,2”,2’”-(1,10-dioxa-4,7,13,16-tetraazacyclooctadecane-4,7,13,16-tetrayl)tetraacetic acid, which is referred to herein as “crown”. The inventors have demonstrated that crown is a novel effective chelator for large metals such as actinium which can coordinate under mild conditions and produce a stable complex in vivo. Further, the in vivo distribution profile of crown when conjugated to a targeting moiety is favourable, indicating good selectivity and specificity for chelation of the desired radiometal. Also, the binding affinity of crown for the desired large radiometals such as actinium is very high relative to currently available chelators, allowing the preparation of an in vivo radioisotope targeting chelate construct having a high specific activity. The preparation of in vivo radioisotope targeting chelate constructs having high specific activity may be particularly important for treatment or prophylaxis of conditions in which the target molecule is expressed at relatively low levels, making the target molecule readily saturable in vivo.

In some embodiments, crown can be directly coupled to a biological targeting moiety, optionally with a linker interposing the crown and the biological targeting moiety, by coupling the biological targeting moiety or linker directly to one of the carboxyl groups of structure (1) to yield the structure shown as (2) below, wherein R₁ is a biological targeting moiety, optionally with a linker interposing the biological targeting moiety and the crown chelator, illustrated as L in structure (3) below.

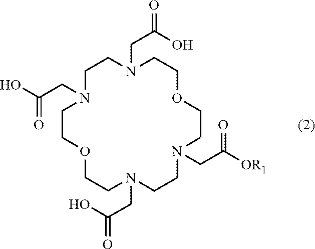

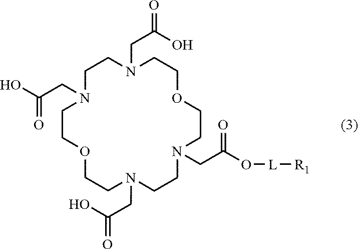

Furthermore, in some embodiments, one or more of the oxygen atoms of the carboxyl group is substituted by a different heterotatom, e.g. N or S. In some embodiments, the crown chelator has the structure shown below as (4), wherein X₁ and X₂ are independently O, N or S. Thus, in various embodiments, the functional group provided on the bifunctional crown chelator to couple the chelator to the biological targeting moiety can be a carboxyl, an ester, an amide, an imide, a thioamide, a thioester, a guanidinium, or the like to yield the structure shown below as (5), wherein R₁ is a biological targeting moiety, optionally with a linker interposing the biological targeting moiety and the crown chelator, illustrated as L in structure (6) below.

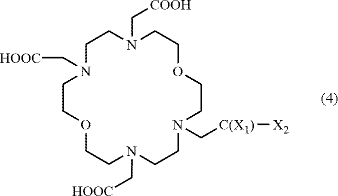

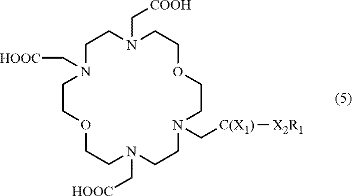

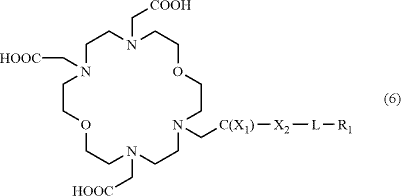

In some embodiments, the crown chelator is provided as a bifunctional chelator, i.e. a chelator bearing an additional functional group that can be used to couple the chelator to a targeting moiety rather than using one of the free carboxyl groups. Any suitable functional group can be coupled to structure (1) at any suitable position to yield a bifunctional chelator. For example, in some embodiments, the bifunctional chelator has the following structure (7), wherein a functional group that can be used to couple the bifunctional chelator a biological targeting moiety to yield the structure below can be provided at one of the positions indicated by R₂, R₃, R₄, R₅ or R₆, wherein R₁ when present in structure (8) or (9) is a biological targeting moiety, optionally with a linker interposing the chelator and the biological targeting moiety, illustrated as L in structure (9) below. Examples of functional groups that can be used for R₂, R₃, R₄, R₅ or R₆ include a carboxyl, an ester, an amide, an imide, a thioamide, a thioester, a guanidinium, an ether, a thioether, an amine, or the like.

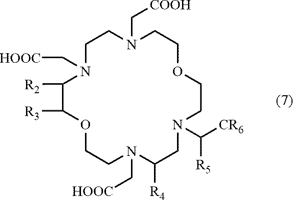

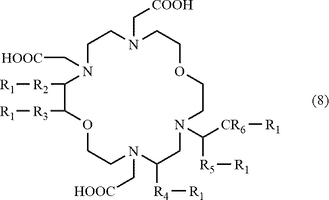

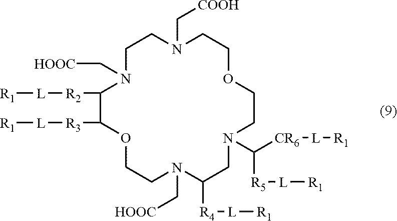

In one example embodiment, bifunctional crown has the following structure (10)

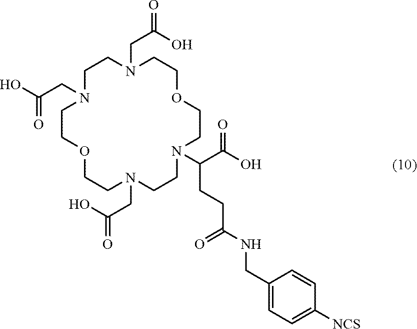

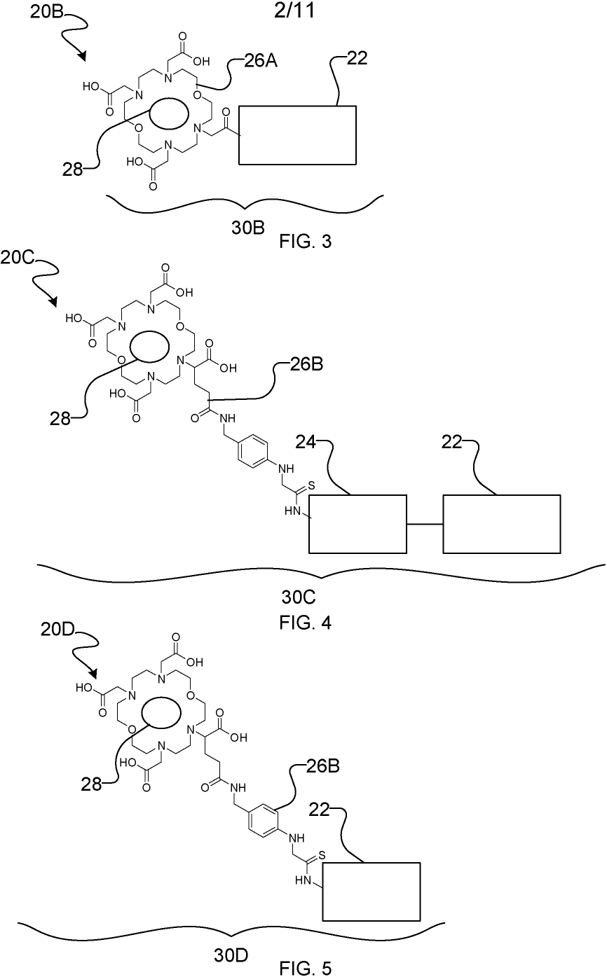

In one example embodiment, with reference to FIGS. 2, 3, 4 and 5, example in vivo targeting chelate constructs illustrated schematically as 20A, 20B, 20C or 20D (collectively referred to herein as in vivo targeting chelate construct 20) have a targeting moiety 22 coupled to a chelator 26A or bifunctional chelator 26B (collectively referred to herein as chelator 26). In some embodiments including the illustrated embodiment of FIGS. 2 and 3, chelator 26A has structure (1) while in other embodiments chelator 26A can be replaced by a chelator having structure (4). In some embodiments, chelator 26B has structure (10) (as in the illustrated embodiment of FIGS. 4 and 5) while in other embodiments chelator 26B can be replaced by a chelator having structure (7). In some other embodiments, structure (2) corresponds to in vivo targeting construct 30B while structure (5) corresponds to in vivo targeting construct 30B having a different crown chelator substituted for chelator 26A. In some other embodiments, structure (3) corresponds to in vivo targeting construct 30A and structure and (6) corresponds to in vivo targeting construct 30A with different crown chelators substituted for chelator 26A. In some other embodiments, structure (8) corresponds to in vivo targeting construct 30D and structure (9) corresponds to in vivo targeting construct 30C, with different bifunctional crown chelators substituted for chelator 26B.

In some embodiments, including the illustrated embodiment of FIGS. 2 and 4, the targeting moiety 22 is coupled to chelator 26 via a suitable linker 24 to yield an in vivo targeting construct 30A or 30C (collectively referred to together with in vivo targeting constructs 30B and 30D as in vivo targeting construct 30). In some embodiments, including the illustrated embodiment of FIGS. 3 and 5, no linker is used and chelator 26 is coupled directly to targeting moiety 22 to yield the in vivo targeting construct 30B or 30D. Chelator 26 can be used to chelate a radionuclide 28 to in vivo targeting construct 30 to yield a metal chelate construct referred to as in vivo targeting chelate construct 20 suitable for targeted in vivo delivery of the radionuclide 28 payload as assisted by targeting moiety 22.

Any moiety suitable for directing the targeted delivery of in vivo targeting chelate construct 20 in vivo can be used as targeting moiety 22 or R₁. In some embodiments, the targeting moiety 22 of the targeting construct 20 is a hapten, antigen, aptamer, affibody molecule, enzyme, protein, peptide, antibody, antigen-binding fragment of an antibody, peptidomimetic, receptor ligand, steroid, hormone, growth factor, cytokine, molecule that recognizes cell surface receptors (including molecules involved in growth, metabolism or function of cells), lipid, lipophilic group, carbohydrate, or any other molecule or targeting component capable of selectively directing a construct to a specific location within the body. The targeting moiety can be produced in any suitable manner, e.g. as a biologic, semisynthetically, or synthetically.

Examples of targeting moieties that have been developed to deliver radioisotope targeting constructs to desired locations within the body of a mammalian subject in vivo include antibodies targeting specific markers associated with specific types of cancers, peptidomimetics targeting proteins that are highly expressed in cancer cells, and the like. Exemplary non-limiting examples of suitable targeting moieties are listed in Table 1.^{[45]} Some targeting moieties selectively interact with biological targets, including antigens, proteins, carbohydrates or other molecules present on the surface of cells that are overexpressed in cancer cells relative to normal cells, e.g. tumor-associated antigens. Exemplary non-limiting examples of suitable targets are listed in Table 1. Suitable targets and/or targeting moieties for radiopharmaceuticals, whether now known or discovered or developed in the future, would be known to a person skilled in the art. In some embodiments, targeting moiety 22 is an antibody or an antigen-binding fragment of an antibody. In some embodiments, targeting moiety 22 is a peptidomimetic. In some embodiments, the targeting moiety 22 is one of the targeting moieties listed in Table 1, with any chelator present in the referenced molecule replaced by a crown chelator. In some embodiments, the targeting moiety 22 interacts selectively with one of the targets listed in Table 1.

TABLE 1

Exemplary targeting moieties and biological targets for targeted radiation therapy.

| | | |
|---|---|---|
| Targeting Moiety | Biological Target | Indications |
| A33 antibody | A33 transmembrane glycoprotein | colorectal cancer |
| dihydrotestosterone (DHT) | androgen receptor (AR) | prostate cancer |
| HuMab-5B1 | CA19.9 | pancreatic cancer, bladder cancer |
| Girentuximab | carbonic anhydrase 9 (CA-IX) | clear cell renal cell carcinoma |
| AMG211 bispecific T-cell engager | carcinoembryonic antigen | gastrointestinal adenocarcinoma |
| IAB22M2C minibody | CD8 | melanoma, lung cancer, hepatocarcinoma |
| Rituximab | CD20 | B-cell lymphoma |
| obinutuzumab | CD20 | B-cell lymphoma |
| Use antibody | CD44v6 | head and neck cancer |
| Plerixafor | C-X-C chemokine receptor type 4 (CXCR4) | hematological and solid malignancies |
| Pentixafor | CXCR4 | hematological and solid malignancies |
| NFB | CXCR4 | hematological and solid malignancies |
| Ipilimumab | Cytotoxic T-lymphocyte-associated protein 4 (CTLA-4) | Melanoma |
| erlotinib | epidermal growth factor receptor (EGFR) | nonsmall cell lung carcinoma; colorectal cancer |
| PD153035 | EGFR | nonsmall cell lung carcinoma; colorectal cancer |
| Afatinib | EGFR | nonsmall cell lung carcinoma; colorectal cancer |
| Cetuximab | EGFR | nonsmall cell lung carcinoma; colorectal cancer |
| panitumumab | EGFR | nonsmall cell lung carcinoma; colorectal cancer |
| ABY-025 affibody | Epidermal growth factor receptor 2 (ERBB2) | breast cancer |
| HER2-nanobody | ERBB2 | breast cancer |
| Trastuzumab | ERBB2 | breast cancer |
| Pertuzumab | ERBB2 | breast cancer |
| GSK2849330 | epidermal growth factor receptor 3 (ERBB3) | solid malignancies |
| lumretuzumab | ERBB3 | solid malignancies |
| 4FMFES | estrogen receptor (ER) | breast cancer and gynecologic cancers |
| FAPI-04 | fibroblast activation protein α | solid malignancies |
| FAPI-21 | fibroblast activation protein α | solid malignancies |
| FAPI-46 | fibroblast activation protein α | solid malignancies |
| Galactose | galactose metabolism | hepatocarcinoma |
| CB-TE2A-AR06 peptide (with crown substituted for DOTA) | gastrin-releasing peptide receptor (GRPR) | prostate cancer, breast cancer, glioma |
| BAY 864367 peptide (with crown-bound ligand label instead of 18F labeling) | GRPR | prostate cancer, breast cancer, glioma |
| RM2 peptide (with crown substituted for DOTA) | GRPR | prostate cancer, breast cancer, glioma |
| SB3 peptide (with crown substituted for DOTA) | GRPR | prostate cancer, breast cancer, glioma |
| RM26 peptide | GRPR | prostate cancer, breast cancer, glioma |
| BBN-RGD peptide | GRPR | prostate cancer, breast cancer, glioma |
| Aca-BBN peptide | GRPR | prostate cancer, breast cancer, glioma |
| NeoBOMB1 peptide (with crown substituted for DOTA) | GRPR | prostate cancer, breast cancer, glioma |
| exendin-4 peptide | glucagon-like peptide 1 receptor (GLP-1R) | insulinoma |
| Glucose | glucose metabolism | neoplasm |
| Codrituzumab | glypican 3 | hepatocarcinoma |
| EF5 | Hypoxia | solid malignancies |
| MISO | Hypoxia | solid malignancies |
| AZA | Hypoxia | solid malignancies |
| HX4 | Hypoxia | solid malignancies |
| ASTM | Hypoxia | solid malignancies |
| LLP2A peptidomimetic | integrin α4β1 | multiple myeloma |
| galacto-RGD peptide | integrin αvβ3 | solid malignancies |
| FPP(RGD)2 peptide | integrin αvβ3 | solid malignancies |
| RGD-K5 peptide | integrin αvβ3 | solid malignancies |
| Fluciclatide | integrin αvβ3 | solid malignancies |
| alfatide-I | integrin αvβ3 | solid malignancies |
| alfatide-II | integrin αvβ3 | solid malignancies |
| PRGD2 peptide | integrin αvβ3 | solid malignancies |
| αvβ6-BP peptide | integrin αvβ6 | head and neck cancer, lung cancer, colorectal cancer, breast cancer, pancreatic cancer |
| CycMSHhex targeting peptides | melanocortin-1 receptor (MC1R) | melanoma |
| MMOT0530A antibody | Mesothelin | pancreatic ductal adenocarcinoma and ovarian cancer |
| SP peptide | Neurokinin1 receptor (NK1R) | glioma |
| neurotensin | Neurotensin 1 receptor (NTS1R) | prostate cancer |
| PARPi | poly(ADP-ribose) polymerase 1 (PARP1) | head and neck cancer |
| PSMA peptidomimetics, e.g. PSMA-1007, PSMA-7 (with crown-bound ligand replacing 18F labelling); PSMA-11 (with crown replacing the HBED chelator); PSMA-617, PSMA-I&T (with crown replacing the DOTA chelator) | Prostate-specific membrane antigen (PSMA) | prostate cancer |
| DCFPyL | PSMA | prostate cancer |
| DCFBC | PSMA | prostate cancer |
| HuJ591 antibody | PSMA | prostate cancer |
| durvalumab | programmed cell death protein (PD-1) | nonsmall cell lung carcinoma |
| nivolumab | PD-1 | nonsmall cell lung carcinoma |
| pembrolizumab | PD-1 | nonsmall cell lung carcinoma |
| BMS-986192 adnectin | programmed death-ligand 1 (PD-L1) | nonsmall cell lung carcinoma, bladder cancer, breast cancer |
| atezolizumab | PD-L1 | nonsmall cell lung carcinoma, bladder cancer, breast cancer |
| MSTP2109A antibody | six-transmembrane epithelial antigen of prostate-1 (STEAP1) | prostate cancer |
| TATE peptide (octreotate) | somatostatin receptor 2 (SSTR2) | neuroendocrine tumors |
| TOC peptide | SSTR2 | neuroendocrine tumors |
| NOC peptide | SSTR2 | neuroendocrine tumors |
| JR11 | SSTR2 | neuroendocrine tumors |
| thymidine | thymidine kinase (DNA replication) | solid malignancies |
| fresolimumab | transforming growth factor-beta (TGF-β) | glioma |
| bevacizumab | vascular endothelial growth factor receptor (VEGFR) | solid malignancies |

Any suitable linker can be used as linker 24 or L to couple chelator 26 to targeting moiety 22 or R₁. For example and by way of illustration only, suitable linkers can include:
a hydrocarbon linker containing between 1 and 10 carbon atoms (C₁-C₁₀), including 2, 3, 4, 5, 6, 7, 8 or 9 carbon atoms that is optionally saturated or unsaturated, optionally substituted with one or more heteroatoms or having one or more substituents; the hydrocarbon linker can be linear, cyclic and/or branched, e.g. 8-aminooctanoic acid, 6-aminohexanoic acid;
an aromatic linker containing an aromatic moiety such as a benzyl group, e.g. aminophenylacetic acid;
an amino acid linker having between 1 and 10 amino acid residues, including 2, 3, 4, 5, 6, 7, 8, or 9 amino acid residues, any one or more of which may be naturally occurring amino acid residues, D-amino acid residues or other non-naturally occurring residues, examples of which include GlyGly (SEQ ID NO:3), GluGluGlu (SEQ ID NO:4), GlySerGlySer (SEQ ID NO:5);
a cyclized linker, or cyclized ring structure, optionally a cyclized amino acid linker, e.g. aminocyclohexanecarboxylic acid;
a PEG-linker of any suitable length;
cationic linkers, whether formed from amino acid residues or other residues, e.g.. Pip, 4-(2-aminoethyl)-1-carboxymethyl-piperazine (Acp);
anionic linkers, whether formed from amino acid residues or other residues, e.g.. AspAsp (SEQ ID NO:6), GluGlu (SEQ ID NO:7);
a carbohydrate containing linker;
click chemistry linkers (triazoles);
any other suitable linker;
or combinations or modifications of the foregoing.
Examples of linkers that have been developed in the art for other radiopharmaceutical targeting constructs are known to those skilled in the art. Hydrophilic or charged linkers such as PEG-linkers or cationic/anionic linkers may be used to increase the overall water solubility of the targeting construct. Amino acid side chain substitutions and/or inclusion of carbohydrate moieties may be made to improve or alter the solubility and/or pharmacokinetics of the targeting construct A person skilled in the art could develop and optimize a suitable linker for a particular application if desired. Examples of linkers that have been developed in the art for other radiopharmaceutical targeting constructs are described, by way of example only and without limitation, by Benešová et al., Barnaski et al. and Kuo et al.^{[41,42,43,44]} A person skilled in the art could develop and optimize a suitable linker for a particular application.

In some embodiments, a construct such as construct 20 is prepared by carrying out suitable reactions to couple targeting moiety 22 and chelator 26, for example via suitable chemical reaction, to yield an in vivo targeting construct 30, optionally with linker 24 interposing targeting moiety 22 and chelator 26. The radionuclide 28 is then added and bound to chelator 26, e.g. at a later time and in a hospital or clinic setting, to form the desired in vivo targeting metal chelate construct 20. In other embodiments, radionuclide 28 could be first chelated with chelator 26, and then chelator 26 is conjugated with targeting moiety 22 in any suitable manner to yield in vivo targeting chelate construct 20.

In some embodiments, the radionuclide 28 is bound to chelator 26 (including as part of construct 30) under mild temperature conditions, e.g. less than about 65° C., 60° C., 55° C., 50° C., 45° C., 40° C., 35° C. or 30° C. In some embodiments, the mild temperature conditions are between about 10° C. and 65° C., including any value or subrange therebetween, e.g. 15° C., 16° C., 17° C., 18° C., 19° C., 20° C., 21° C., 22° C., 23° C., 24° C., 25° C., 26° C., 27° C., 28° C., 29° C., 30° C., 35° C., 40° C., 45° C., 50° C., 55° C. or 60° C. In some embodiments, the radionuclide 28 is conjugated to chelator 26 or construct 30 at room temperature, i.e. in the range of about 15° C. to about 25° C., including any temperature value therebetween, e.g. 16° C., 17° C., 18° C., 19° C., 20° C., 21° C., 22° C., 23° C., or 24° C.

In some embodiments, the radionuclide 28 or construct 30 is combined with chelator 26 to form a metal chelate under mild pH conditions, e.g. between about 5.0 and about 7.4, including any value or subrange therebetween, e.g. 5.2, 5.4, 5.6, 5.8, 6.0, 6.2, 6.4, 6.6, 6.8, 7.0 or 7.2. In some embodiments the radionuclide 28 is conjugated to chelator 26 at approximately neutral pH, i.e. a pH of approximately 7.0, e.g. between about 6.8 and 7.2 including any value therebetween, e.g. 6.9, 7.0 or 7.1. In some embodiments, the radionuclide 28 is conjugated to chelator 26 at approximately physiological pH, i.e. at approximately pH 7.4, e.g. between about 7.2 and 7.6 including any value therebetween, e.g. 7.3, 7.4 or 7.5. In some embodiments, radionuclide 28 is combined with chelator 26 or construct 30 in aqueous solution. In some embodiments, the aqueous solution is free or substantially free of alcohol such as ethanol.

In some embodiments, the radionuclide 28 is combined with chelator 26 or construct 30 for an incubation period to allow a chelated metal complex to form. In some embodiments, the incubation period is between about 5 minutes and about 6 hours, including any period therebetween, e.g. 10, 15, 20, 25, 30, 45, 60 or 90 minutes, or 2, 3, 4 or 5 hours. In some embodiments, the incubation period is between about 5 minutes and about 30 minutes.

In some embodiments, the concentration of chelator 26 or construct 30 that is present when conjugated to radionuclide 28 is between about 10⁻⁴ to 10⁻⁷M, including any value therebetween, e.g. 10⁻⁵ or 10⁻⁶M. The concentration of chelator 26 or construct 30 that is used can be adjusted depending on the complexation kinetics between the particular chelator 26 and radionuclide 28 used in any particular embodiment. Similarly the temperature at which the radionuclide 28 is combined with chelator 26 or construct 30 can be varied depending on the complexation kinetics.

In some embodiments, in vivo radioisotope targeting chelate construct 20 is present in mammalian serum, optionally in human serum. In some embodiments, in vivo radioisotope targeting chelate construct 20 is stable in mammalian serum, optionally in human serum. In some embodiments, in vivo radioisotope targeting chelate construct 20 is present in mammalian serum within the body of a mammal, optionally in human serum within the body of the human. In some embodiments, in vivo radioisotope targeting chelate construct 20 is present in mammalian blood, optionally in human blood. In some embodiments, in vivo radioisotope targeting chelate construct 20 is present in mammalian blood within the body of a mammal, optionally in human blood in the body of the human. In some embodiments, in vivo radioisotope targeting chelate construct 20 is present within the body of a mammal, optionally the body of a human. In some embodiments, in vivo radioisotope targeting chelate construct 20 is present in a mammalian cell, optionally a human cell.

In some embodiments, radionuclide 28 is delivered to a selected location within the body of a mammalian subject by administering to the subject an in vivo radioisotope targeting chelate construct 20 incorporating the radionuclide 28 and a targeting moiety 22 that specifically directs the in vivo radioisotope targeting chelate construct 20, including the bound radionuclide 28, to the selected location within the body of the subject. In some embodiments, the method includes allowing the targeting moiety 22 to enhance the accumulation of the in vivo radioisotope targeting chelate construct 20 at the selected location within the body relative to other locations in the body to selectively deliver a dose of radiation to the selected location. In some embodiments, the in vivo radioisotope targeting chelate construct 20 is used to cause cell death at the selected location by delivering a targeted dose of radiation. In some embodiments, the cells that are killed at the selected location are cancer cells. In some embodiments, the radiation is alpha radiation.

In some embodiments, in vivo radioisotope targeting chelate construct 20 is internalized by a cell within the mammalian subject, for example by endocytosis or otherwise. Thus in some embodiments, in vivo radioisotope targeting chelate construct 20 is present within a mammalian cell. In some embodiments, the in vivo radioisotope targeting chelate construct 20 is present within a human cell.

In some embodiments, the in vivo radioisotope targeting chelate construct 20 is prepared prior to administration of construct 20 to a subject by combining an in vivo radioisotope targeting construct 30 having a targeting moiety 22, a chelator 26 and optionally a linker 24 with a radionuclide 28 to form the in vivo radioisotope targeting chelate construct 20. In some embodiments, the combining is carried out at a mild temperature, e.g. at a temperuature in the range of about 10° C. to about 65° C., including any value therebetween e.g. 15° C., 16° C., 17° C., 18° C., 19° C., 20° C., 21° C., 22° C., 23° C., 24° C., 25° C., 26° C., 27° C., 28° C., 29° C., 30° C., 35° C., 40° C., 45° C., 50° C., 55° C. or 60° C. In some embodiments, the combining is carried out at a mild pH, e.g. an approximately neutral pH or an approximately physiological pH. In some embodiments, the mild pH is a pH of between about 5.0 and about 7.4, including any value therebetween e.g. 5.2, 5.4, 5.6, 5.8, 6.0, 6.2, 6.4, 6.6, 6.8, 7.0 or 7.4. In some embodiments, the mild pH is approximately 6.0. In some embodiments, the combining is carried out a physiological pH, e.g. in the range of about. 7.0 to 7.4 including any value therebetween, e.g. 7.1, 7.2 or 7.3. In some embodiments, radionuclide 28 is combined with in vivo radioisotope targeting construct 30 in aqueous solution. In some embodiments, the aqueous solution is free or substantially free of alcohols such as ethanol. In some embodiments, the combining is carried out for a period of between about 5 and about 30 minutes, including any value therebetween e.g. 10, 15, 20 or 25 minutes.

In some embodiments, in vivo targeting chelate construct 20 is used in diagnostic applications. For example, in vivo targeting chelate construct 20 may be administered to a subject in any suitable manner, and any suitable imaging technology or procedure may be used to evaluate the localization of the targeting chelate construct 20 within the body via targeting moiety 22 by visualizing the location of bound radionuclide 28, e.g. positron emission tomography (PET) imaging or single-photon emission computerized tomography (SPECT) imaging. Such imaging procedures can be carried out for example to diagnose a subject as having a particular disorder or type of cancer, or to localize regions of the subject’s body affected by the particular disorder or type of cancer. In some embodiments, localization of targeting chelate construct 20 to a target organ, region or plurality of loci within the body as evaluated by such imaging technology may be indicative that the subject has a particular form of cancer, and/or can be used to evaluate the extent of the cancer and or locations within the body wherein cancerous cells are or may be located, and/or can be used to evaluate the extent of metastasis of the cancer.

In some embodiments, constructs such as targeting chelate construct 20 are used in therapeutic applications, for example to carry out targeted radionuclide therapy. For example, targeting chelate construct 20 may be administered to a subject in any suitable manner, and the targeting effect imparted by targeting moiety 22 can be used to deliver the chelated radionuclide 28 to a desired location within the subject’s body. In some embodiments, radiation from radionuclide 28 is used to kill cells at the desired location. In some embodiments, the cells that are killed at the desired location are cancer cells. In some embodiments, targeting construct 20 is used to perform targeted radionuclide therapy. In some embodiments, targeting construct 20 is used to perform targeted alpha therapy.

In some embodiments, a pharmaceutical composition is provided, the pharmaceutical composition comprising a construct such as targeting construct 20 and a pharmaceutically acceptable carrier. The pharmaceutical composition may include any suitable excipient, vehicle, buffer, diluent, binder, thickener, lubricant, preservative or the like, and may be provided in any desired state, e.g. as a liquid, suspension, emulsion, paste, or the like. In some embodiments, the pharmaceutical composition can be administered in any suitable manner, e.g. orally, intravenously, intramuscularly, subcutaneously, intraperitoneally, intratumorally, by inhalation, or the like.

In some embodiments, a method of prophylaxis and/or treatment of a subject having or believed to have cancer is provided. In some embodiments, the method comprises administering an in vivo targeting chelate construct 20 or a pharmaceutical composition comprising such a targeting chelate construct 20 to the subject. In some embodiments, the method comprises administering a therapeutically and/or prophylactically effective amount of the targeting chelate construct 20 to the subject.

In some embodiments, the subject is a mammal. In some embodiments, the subject is a human. In alternative embodiments, the subject is livestock or a pet, e.g. a horse, cow, sheep, goat, cat, dog, rabbit, or the like. In some embodiments, the subject is a monkey.

While exemplary embodiments are described herein with reference to the targeting and killing of cancer cells, such constructs can be used for the selective killing and/or ablation of other undesired cell types, for example bacteria, fungi, cells implicated in autoimmune disorders, virus-infected cells, parasites, and so on.

In some embodiments, the metals that can be used as metal 28 include actinides, lanthanides, rare earth metals, or main group metals. In some embodiments, the lanthanide is La, Ce, Pr, Nd, Pm, Sm, Eu, Gd, Tb, Dy, Ho, Er, Tm, Yb or Lu. In some embodiments, the lanthanide is Gd, Lu, Pr, Nd, Ho, Er or Yb. In some embodiments, the lanthanide is a radiolanthanide. In some embodiments, the actinide is Ac, Th, Pa, U, Np, Pu, Am, Cm, Bk, Cf, Es, Fm, Md, No or Lr. In some embodiments, the actinide is Ac, Th or U. In some embodiments, the actinide is a radioactinide. In some embodiments, the rare earth metal is Sc, Y, La, Ce, Pr, Nd, Pm, Sm, Gd, Tb, Dy, Ho, Er, Tm, Yb or Lu.

In some embodiments, the metal is a radioisotope. In some embodiments, the radioisotope is any desired radioisotope, e.g. ²²⁵Ac, ²²⁷Th, ²²⁶Th, ²¹¹At, ⁴⁴Sc, ⁹⁰Y, ⁸⁹Zr,¹⁷⁷Lu, ¹¹¹ln, ^{86/89/90}Y ²¹¹At, ²¹¹Fr, ^{212/213}Bi, ¹⁵³Sm ^{161/166}Ho, ^{165/166}Dy, ^{161/155}Tb, ¹⁴⁰La, ^{142/143/145}Pr, ¹⁵⁹Gd, ^{169/175}Yb, ^{167/170}Tm ¹⁶⁹Er, ¹⁴⁹Pm, ¹⁵⁰Eu, ⁶⁸Ga, ¹³⁷CS, ¹⁴¹Ce, or the like.

In some embodiments, the metal is actinium (Ac), lutetium (Lu), bismuth (Bi), gallium (Ga), indium (In), terbium (Tb), thorium (Th), or Caesium (Cs). In some embodiments, the metal is actinium (III) (Ac³⁺), lutetium (III) (Lu³⁺), bismuth (III) (Bi³⁺), gallium (III) (Ga³⁺), indium (III) (ln³⁺), terbium (Tb³⁺), thorium (III) (Th³⁺), or Cesium (I) (Cs¹⁺) . In some embodiments, the metal is ²²⁵Ac, ¹⁷⁷Lu, ²¹³Bi, ²³²Th, ²³⁰Th, ²²⁸Th, ⁶⁸Ga, ¹⁶¹Tb, ¹⁵⁵Tb, ¹⁵²Tb, ¹⁴⁹Tb, ¹¹¹ln, or ¹³⁷CS.

In some embodiments, crown is bound to a metal ion to form a coordination complex. In some embodiments, the coordination complex is referred to as a metal chelate. In some embodiments, the metal chelate or crown as the chelating ligand is associated with one or more cations as counter ions, for example Na⁺, K⁺, Ca²⁺ or the like. In some embodiments, the metal chelate or the chelating ligand is fully protonated. In some embodiments, the metal chelate or the chelating ligand is in its free acid form. In some embodiments, the metal chelate or the chelating ligand is in a partially protonated state.

In some embodiments, the coordination complex is present in mammalian serum, optionally human serum. In some embodiments, the coordination complex is stable in mammalian serum, optionally human serum. In some embodiments, the coordination complex is present in mammalian serum within the body of the mammal, optionally present in human serum within the body of the human. In some embodiments, the coordination complex is present in blood, optionally human blood. In some embodiments, the coordination complex is stable in mammalian blood, optionally human blood. In some embodiments, the coordination complex is present in mammalian blood within the body of the mammal, optionally present in human blood within the body of the human. In some embodiments, the coordination complex is present within the body of a mammal, optionally present within the body of a human. In some embodiments, the coordination complex is present within a cell of a mammalian subject, optionally present within a cell of a human subject.

Without being bound by theory, the examples described herein demonstrate that crown as a chelator has a high binding affinity for binding radiometals, particularly larger radiometals, including the exemplary radiometals ²²⁵Ac, ²¹³Bi, ¹⁷⁷Lu, ¹⁵⁵Tb and ⁶⁸Ga. The high binding affinity of crown for such exemplary radiometals is demonstrated for example by the ability of crown to form coordination complexes with the radiometals quantitatively at room temperature conditions and neutral pH at chelator concentrations as low as 10⁻⁵ M or 10⁻⁶ M, as compared with the current gold standard chelator DOTA which requires higher concentrations on the order of 10⁻⁴ M and harsher chelation conditions of 90° C. for 30 minutes to obtain a similar degree of labelling, which is too harsh for many biological targeting moieties (e.g. antibodies) to withstand. This high binding affinity allowed for example the generation of an in vivo targeting chelate construct incorporating ²²⁵Ac with a molar activity (specific activity) of 4.1 MBq/nmol, considerably higher than the parallel preparation previously generated using DOTA which was able to chelate ²²⁵Ac with a molar activity of only approximately 200 kBq/nmol.^{[29]} This difference in specific activity allows for the accumulation of a higher uptake of the radiometal in the tumor tissue as compared with other tissues. Thus, in some embodiments, a radioisotope targeting construct incorporating crown as a chelator has a specific activity of at least 4 MBq/nmol.

Without being bound by theory, the significantly higher specific activity of the in vivo targeting chelate construct may be particularly important where the construct is used against a target with relatively low levels of expression in vivo, which means that the target can be readily saturated by in vivo targeting construct molecules that are not bound to the radiometal, thereby blocking effective delivery of the radiometal to its desired locus of administration. Thus, crown is expected to be more effective against targets with low levels of expression in vivo where current chelators do not work well for conducting targeted radiotherapy.

Furthermore, the inventors found that crown effectively chelated the desired radiometals with good stability over several days at 37° C. in human serum, and further the biological distribution profile of the exemplary tested in vivo targeting chelate constructs demonstrated good selective accumulation in tumor tissue as compared with normal tissues - if the crown-radiometal complex were unstable in vivo, then it would be expected to observe accumulation of the radiometal in the blood, liver and spleen, as has been observed for administration of free ²²⁵Ac.^{[19]} In contrast, the inventors observed accumulation of the radiometal only in the clearance track for the tested in vivo radioisotope targeting chelate constructs (i.e. in the renal pathway, kidney, urine and bladder), indicating that crown effectively retained the bound radiometal when administered in vivo.

Thus, from the examples described herein, it can be soundly predicted that crown can be used as a chelator for the in vivo delivery of radioisotopes for the conduct of targeted radiotherapy or imaging when conjugated to a targeting moiety that targets the vector to a suitable location in vivo.

### EXAMPLES

Specific embodiments are further described with reference to the following examples, which are intended to be illustrative and not limiting in scope.

### Example 1.0 - Synthesis and Characterization of Crown

Crown having the structure (1) was synthesized according to Scheme 1, which shows the synthesis of 2,2’,2”,2”’-(1,10-dioxa-4,7,13,16-tetraazacyclooctadecane-4,7,13,16-tetrayl)tetraacetic acid (“crown”).

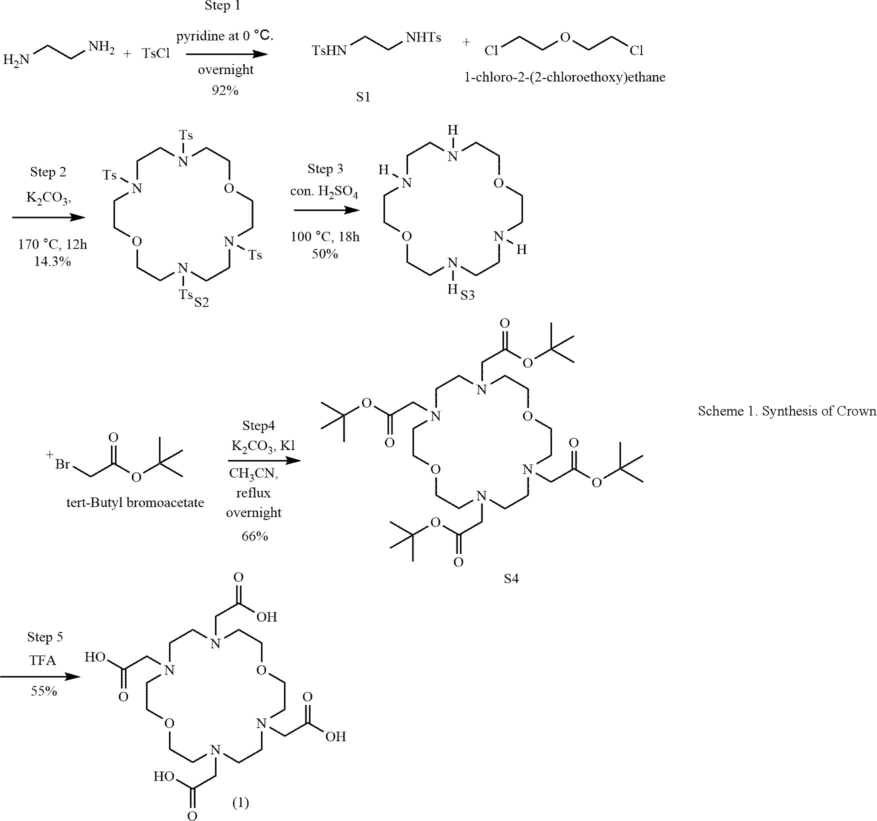

S1, S2, S3 were synthesized using reported methods. ^{[28]} S4 was synthesized from adapting reported method.^{[27]}

Preparation of N,N′-(ethane-1,2-diyl)bis(4-methylbenzenesulfonamide) S1: p-toluenesulfonyl chloride (7.8 g, 40.5 mmol) was added to a stirred solution of ethylenediamine (1.33 mL. 20 mmol) in 100 mL pyridine at 0° C. After overnight stirring at room temperature, the mixture was poured into 250 mL water. The resulting precipitate were filtered, washed with diethyl ether, and dried to obtain the title compound (6.8 g, 92.4 %) as a white powder. ¹H NMR (300 MHz, DMSO-d₆): δ 7.61 - 7.57 (m, 4 H), 7.36 (d, J = 8.0 Hz, 4 H), 2.73 - 2.65 (m, 4 H), 2.37 (s, 6 H). ¹³ C NMR (151 MHz, CDCl3) δ 165.57, 160.34, 146.90, 137.76, 124.11, 123.83, 77.27, 77.06, 76.85, 64.64, 52.94. ESI MS: [M+Py+H]⁺:448.1

Preparation of 4,7,13,16-tetratosyl-1,10-dioxa-4,7,13,16-tetraazacyclooctadecane S2: N,N′-(ethane-1,2-diyl)bis(4-methylbenzenesulfonamide) (7.4 g, 0.02 mol ), K₂CO₃ (4.2 g, 0.03 mol) and 2-chloroethyl ether (2.35 mL, 0.02 mol) were dissolved in dimethylformamide (20 mL) and heated on an oil bath at 170° C. for 12 h. After cooling to room temperature, 60 mL water was added to the reaction mixture. The precipitate was filtered and washed with water (3 times) and acetone (50 mL). The S2 (2.5 g, 14.3%) was obtained as a white solid after recrystallization of crude product from DMF. ¹H NMR (300 MHz, DMSO-d₆) δ 7.63 (d, J= 8.0 Hz, 8 H), 7.41 (d, J= 8.1 Hz, 8 H), 3.45 (t, J= 5.0 Hz, 8 H), 3.25 - 3.06 (m, 16 H), 2.42 - 2.32 (m, 12 H). ¹³ C NMR (151 MHz, CDCl₃) δ 165.13, 157.36, 147.35, 138.50, 127.33, 124.56, 77.27, 77.06, 76.85, 53.19, 32.89. ESI MS: [M+H]⁺:877.6

Preparation of 1,10-dioxo-4,7,13,16-tetraazacyclooctadecane S3: 4,7,13,16-tetratosyl-1,10-dioxa-4,7,13,16-tetraazacyclooctadecane (2 g, 2.3 mmol) was dissolved in 20 mL concentrated H₂SO₄ and heated at 100° C. for 18 h. Then sodium hydroxide solution was added to the reaction mixture to adjust the pH to 10 - 11. The mixture was extracted with chloroform, dried with anhydrous sodium sulfate and evaporated to give the tetraazacrown ether S3 as a slightly colorless solid (300 mg, 50.2%). ¹H NMR (600 MHz, DMSO-d₆) δ 3.48 - 3.43 (m, 8 H), 2.65 (m, 8 H), 2.60 (s, 8 H). ¹³ C NMR (151 MHz, DMSO-d₆) δ 70.15, 49.46, 49.43. ESI MS: [M+Na]⁺:283.2

Preparation of tetra-tert-butyl 2,2’,2”,2,”-(1,10-dioxa-4,7,13,16-tetraazacyclooctadecane-4,7,13,16-tetrayl)tetraacetate S4: A solution of tert-butyl bromoacetate (176 mg, 0.9 mmol) in dry CH₃CN (5 mL) was added dropwise to a mixture of 1,10-dioxo-4,7,13,16-tetraazacyclooctadecane (52 mg, 0.2 mmol), K₂CO₃ (138 mg, 1 mmol) and KI (7 mg, 0.04 mmol) in dry CH₃CN (10 mL). The resulting mixture was stirred at room temperature for 1h and then refluxed overnight. After cooling to room temperature, the mixture was filtered, and the residue washed with dry CH3CN. The filtrate was collected and evaporated to yield S4 as white solid (95 mg, 66%). ¹H NMR (600 MHz, DMSO-d₆) δ 3.85 (s, 8 H), 3.64 (t, J= 5.0 Hz, 8 H), 3.26 - 3.06 (m, 16 H), 1.46 (s, 36 H). ¹³ C NMR (151 MHz, DMSO-d₆) δ 168.47, 82.64, 67.03, 54.78, 53.57, 50.72, 28.19. HRMS (ESI) calculated for C₃₆H₆₉N₄O₁₀⁺ [M+H]⁺:717.5013, found: 717.5105

Preparation of 2,2’,2”,2”’-(1,10-dioxa-4,7,13,16-tetraazacyclooctadecane-4,7,13,16-tetrayl)tetraacetic acid (1) (Crown): tetra-tert-butyl 2,2’,2’’,2’’’-(1,10-dioxa-4,7,13,16-tetraazacyclooctadecane-4,7,13,16-tetrayl)tetraacetate (105 mg, 0.15 mmol) was dissolved in 10 mL of mixed solvent containing 9.5 mL TFA, 0.25 mL TIPS and 0.25 mL H₂O. The mixture was stirred at room temperature for 2 hours. The solvent was removed by air flow and the residue was dissolved in CH₃CN/H₂O (1/1) for further HPLC purification with the method: 0-5 min 100% H₂O+0.1% TFA, 5-25 min 100%-60% H₂O+0.1% TFA. The fraction at 6.8 min was collected and lyophilized to give crown as colorless oil (40 mg, 55.4%). ¹H NMR (600 MHz, DMSO-d₆) δ 3.86 (s, 8 H), 3.72 - 3.58 (m, 8 H), 3.39 - 3.02 (m, 16 H). ¹³ C NMR (151 MHz, DMSO) δ 170.63, 158.60 (q, J= 34.7 Hz, CF₃CO₂H), 116.68 (q, J= 293.9 Hz, CF₃CO₂H), 66.92, 54.10, 53.63, 50.65. HRMS (ESI) calculated for C₂₀H₃₇ N₄O₁₀⁺ [M+H]⁺:492.2431, found: 492.2502.

### Example 2.0 - Synthesis of Crown-TATE Targeting Construct

The inventors attached crown to a peptide (octreotate (referred to herein as “TATE”), which is a targeting moiety having the amino acid sequence H-D-Phe-Cys-Phe-D-Trp-Lys-Thr-Cys-Thr-OH (SEQ ID NO:1) having the following structure (11):

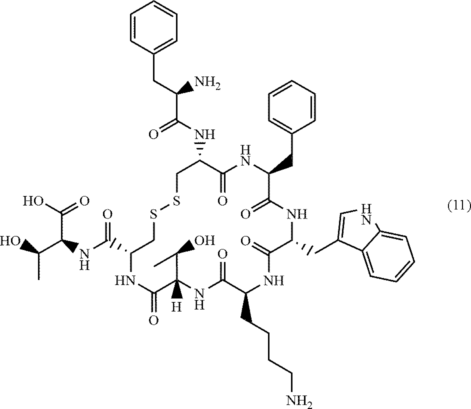 [SMILES: C[C@@H](O)[C@H](NC(=O)[C@@H]1CSSC[C@H](NC(=O)[C@H](N)Cc2ccccc2)C(=O)N[C@@H](Cc2ccccc2)C(=O)N[C@H](Cc2c[nH]c3ccccc23)C(=O)N[C@@H](CCCCN)C(=O)N[C@@H]([C@@H](C)O)C(=O)N1)C(=O)O]

TATE is an SSR agonist that can be used as a targeting moiety to target a construct to SSRs, which are found with high density in various malignancies, including malignancies of the central nervous system, breast, lung and lymphatic system. Ac-crown-TATE has the following structure (12) and crown is directly linked to the free amino group of the N-terminal D-phenylalanine residue of TATE via an amide linkage formed through one of the free carboxylic acid groups of crown:

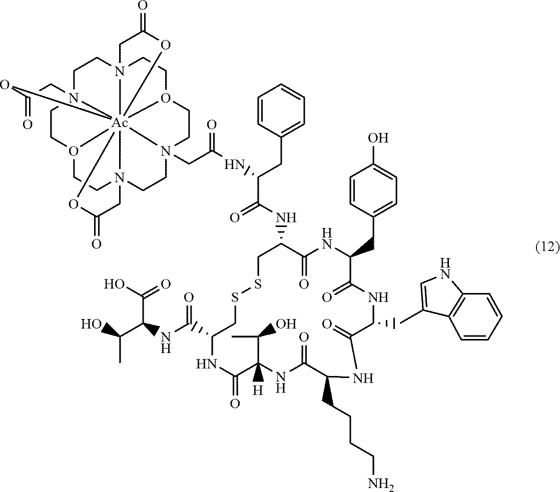

The coupling of crown having structure (1) to uncleaved peptide on resin gave low yield possibly due to the poor solubility. 3tBu-Crown was synthesized to improve the solubility according to Scheme 2.

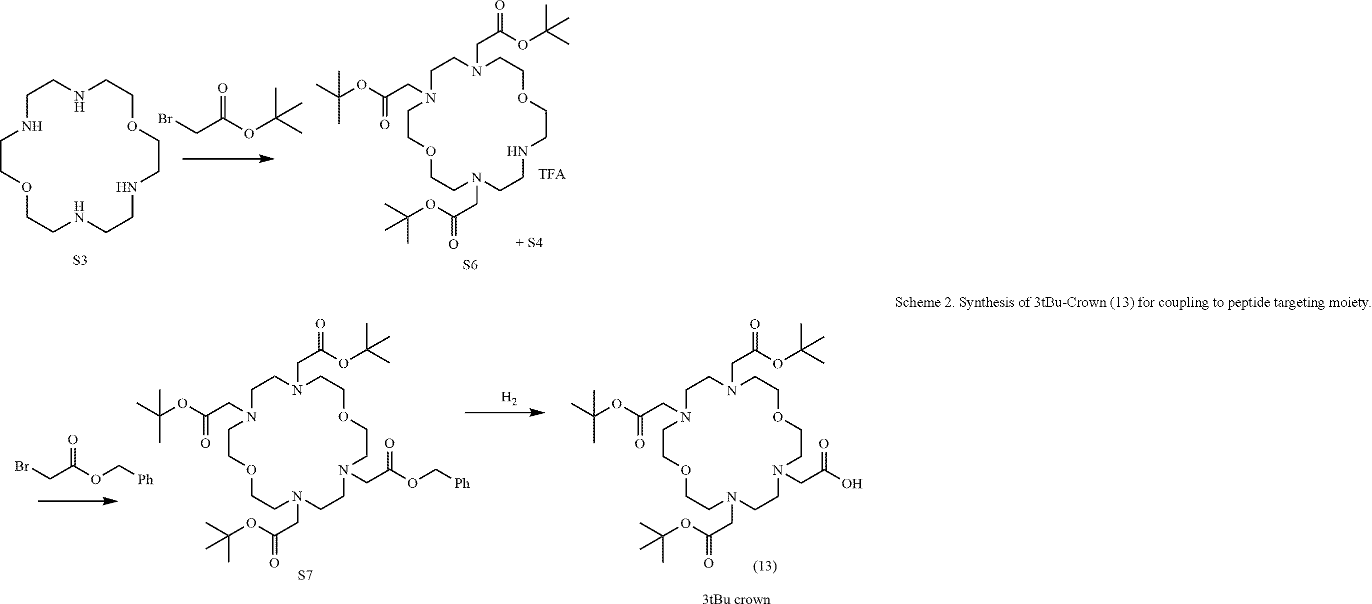

Preparation of tri-tert-butyl 2,2’,2”-(1,10-dioxa-4,7,13,16-tetraazacyclooctadecane-4,7,13-triyl)triacetateTFA salts S6: A solution of tert-butyl bromoacetate (656.8 mg, 3.4 mmol) in dry CH₃CN (2 mL) was added dropwise to a mixture of 1,10-dioxo-4,7,13,16-tetraazacyclooctadecane (265.0 mg, 1.0 mmol), NaHCOs (282.2 mg, 3.4 mmol) in dry CH₃CN (20 mL). The resulting mixture was stirred at room temperature overnight. After filtration, the filtrate was collected and evaporated. The residue was dissolved in CH₃CN/H₂O (2/1) for further HPLC purification with the method: 0-1 min 98% H₂O+0.1 % TFA, 1-10 min 98%-0% H2O+0.1 % TFA. The fraction at 6.8 min was collected and lyophilized to give the title compound as white solid (246.3 mg, 34.0%). Also with S4 (7.4 min, 151.3 mg, 21.1%). ¹HNMR (500 M, CDCl₃):δ 11.95 (brs, 3 H, TFA), 9.19 (brs, 1 H, TFA), 3.93 (s, 2 H), 3.82 (t, J = 4.7 Hz, 2 H), 3.78 - 3.70 (m, 4 H), 3.67 (s, 2 H), 3.56 (t, J = 4.6 Hz, 2 H), 3.50 (t, J= 5.8 Hz, 2 H), 3.46 (t, J= 4.6 Hz, 2 H), 3.42 (s, 2 H), 3.39 (t, J= 5.7 Hz, 2 H), 3.25 - 3.20 (m, 4 H), 3.17 - 3.08 (m, 4 H), 2.95 (t, J= 4.6 Hz, 2 H), 1.44 (s, 9 H), 1.42 (s, 9 H), 1.41 (s, 9 H). ¹³ C NMR (125 MHz, CDCl₃) δ 170.8, 167.8, 166.1, 160.9 (q, J= 36.7 Hz, CF₃CO₂H), 115.9 (q, J = 290.3 Hz, CF₃CO₂H), 84.3, 83.6, 82.8, 67.8, 67.1, 66.2, 65.2, 56.1, 55.2, 54.5, 53.6, 53.2, 51.1, 50.4, 49.9, 47.2, 45.7, 27.8, 27.8, 27.7. RMS (ESI) calcd for C₃₀H₅₉ N₄O₈⁺ [M+H]⁺: 603.4327, found: 603.4312.

Preparation of tri-tert-butyl 2,2’,2”-(16-(2-(benzyloxy)-2-oxoethyl)-1,10-dioxa-4,7,13,16-tetraazacyclooctadecane-4,7,13-triyl)triacetate S7: The mixture of tri-tert-butyl 2,2ʹ,2”-(1,10-dioxa-4,7,13,16-tetraazacyclooctadecane-4,7,13-triyl)triacetate (246.3 mg, 0.34 mmol), benzyl bromoacetate(236.4 mg, 1.03 mmol), triethylamine (208.2 mg, 2.06 mmol) in 15 ml CH₂Cl₂ was heated at 80° C. for reflux overnight. After cooling to room temperature, the solvent was removed rotary evaporation and the residue was dissolved in CH₃CN/H₂O (2/1) for further HPLC purification with the method: 0-1 min 98% H2O+0.1% TFA, 1-10 min 98%-0% H₂O+0.1% TFA. The fraction at 7.4 min was collected and lyophilized to give the title compound as white solid (197.7 mg, 77.4%). ¹H NMR (600 M, CDCl₃): δ 12.27 (brs, 4 H, TFA), 7.39 - 7.28 (m, 5 H), 5.13 (s, 2 H), 3.91 (s, 2 H), 3.81 - 3.68 (m, 12 H), 3.63 (t, J = 4.8 Hz, 2 H), 3.48 - 3.42 (m, 4 H), 3.42 - 3.31 (m, 6 H), 3.29 - 3.23 (m, 4 H), 3.11 (t, J= 4.8 Hz, 2 H), 1.65 - 0.88 (m, 27 H). ¹³ C NMR (150 MHz, CDCl₃) δ 169.4, 167.4, 167.0, 166.2, 160.8 (q, J = 37.1 Hz, CF₃CO₂H), 134.8, 128.6 (two carbons), 128.4, 115.9 (q, J = 290.1 Hz, CF₃CO₂H), 84.4, 84.0, 83.8, 67.5, 67.2, 66.7, 66.3, 65.8, 54.5, 54.4, 54.05, 54.01, 53.98, 53.93, 53.8, 53.7, 51.3, 50.7, 50.6, 50.0, 27.75, 27.74, 27.72. HRMS (ESI) calcd for C₃₉H₆₇ N₄O₁₀⁺ [M+H]⁺: 751.4852, found: 751.4774.

Preparation of 2-(7,13,16-tris(2-(tert-butoxy)-2-oxoethyl)-1,10-dioxa-4,7,13,16-tetraazacyclooctadecan-4-yl)acetic acid (13): The mixture of tri-tert-butyl 2,2’,2”-(16-(2-(benzyloxy)-2-oxoethyl)-1,10-dioxa-4,7,13,16-tetraazacyclooctadecane-4,7,13-triyl)triacetate (197.7 mg, 0.26 mmol), 10% Pd/C (60 mg) in 12 ml MeOH was stirred at H₂ atmosphere via H₂ balloon for 4 hours. After filtration, the filtrate was collected and evaporated to yield the title compound as foamed solid (162.2 mg, 94.5% ). ¹H NMR (600 M, CDCl₃): δ 11.46 (brs, 4 H, TFA and CO₂H), 4.22 - 3.20 (m, 32 H), 1.48 - 1.37 (m, 27 H). ¹³ C NMR (150 MHz, CDCl₃) δ 169.5, 167.0, 166.9, 166.2, 160.9 (q, J= 36.7 Hz, CF₃CO₂H), 116.09 (q, J = 290.8 Hz, CF₃CO₂H), 84.3, 84.0, 83.8, 66.51, 66.46, 65.8, 65.5, 55.0, 54.43, 54.39, 54.30, 54.1, 54.0, 51.2, 50.3, 49.9, 49.7, 27.9, 27.84, 27.82. HRMS (ESI) calculated for C₃₂H₆₁ N₄O₁₀⁺ [M+H]⁺: 661.4382, found: 661.4327.

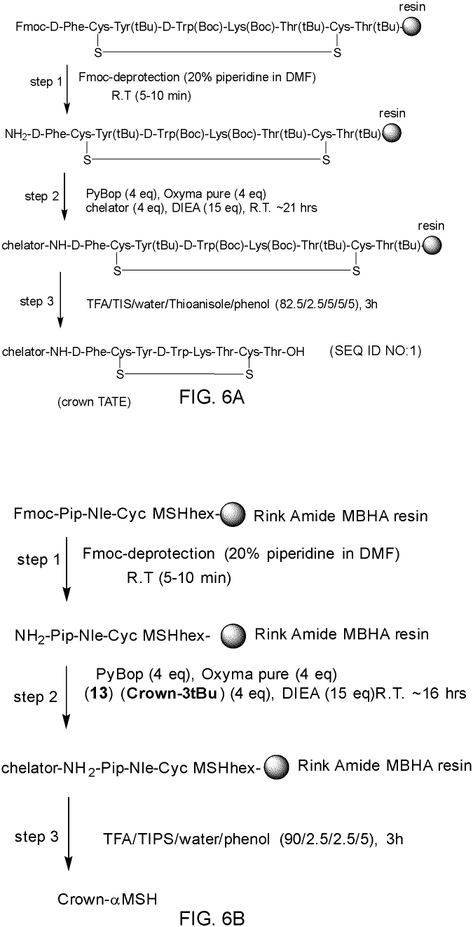

3tBu-Crown was attached to the peptide TATE, using the synthetic scheme (Scheme 3) shown in FIG. 6A.

### Example 3.0 - Preparation of 225Ac-Crown-αMSH

The synthesis of crown is shown above in Scheme 1 above.^{[27,28]} A targeting construct comprising crown coupled to αMSH having the structure (14) below was synthesized as described further below following the scheme (Scheme 4) shown in FIG. 6B. The αMSH targeting moiety has a lactam bridge cyclized α-melanocyte-stimulating hormone core (Ac-Nle⁴-cyclo[Asp⁵-His-D-Phe⁷-Arg-Trp-Lys¹⁰]-NH₂) moiety (SEQ ID NO:2) (also called Nle-CycMSHₕₑₓ) that is coupled at its amino-terminal end to 4-amino-(1-carboxymethl) piperidine (Pip). Crown is linked to the free amino group of the 4-amino-(1-carboxymethyl) piperidine via one of the free carboxylic acid groups of crown, and the 4-amino-1-carboxymethyl) piperidine together with the norleucine residue form a linker to the CycMSHhex moiety.

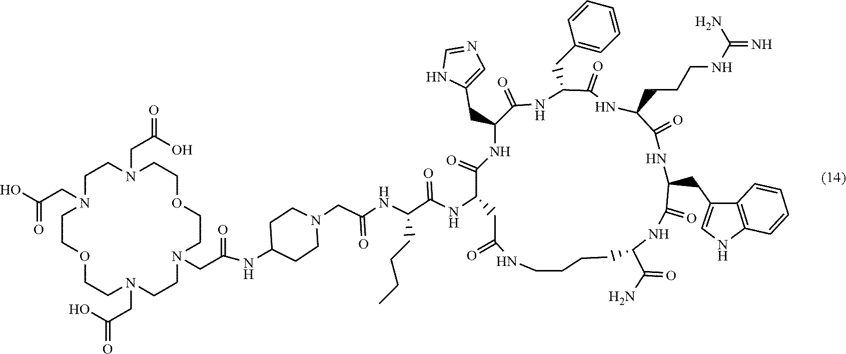 [SMILES: CCCC[C@H](NC(=O)CN1CCC(NC(=O)CN2CCOCCN(CC(=O)O)CCN(CC(=O)O)CCOCCN(CC(=O)O)CC2)CC1)C(=O)N[C@H]1CC(=O)NCCCC[C@@H](C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2cnc[nH]2)NC1=O]

Preparation of crown-αMSH peptide: Peptide synthesis for Fmoc-Pip-Nle-CycMSHₕₑₓ-resin (Fmoc-αMSH-resin) was performed as described in previously published procedures^{[29]} using the scheme (Scheme 4) shown in FIG. 6B. Subsequently, Fmoc was removed. Benzotriazol-1-yl-oxytripyrrolidinophosphonium hexafluorophosphate (PyBOP, 4 eq.), ethyl cyano(hydroxyimino)acetate (Oxyma Pure, 4 eq.) and crown(tBu)₃ (4 eq.) were dissolved in DMF (minimum), then added to the resin (1 eq.) and initiated by the addition of N,N-diisopropylethylamine (DIEA, 15 eq.). Coupling was carried out for ~21 h at room temperature with shaking. The resin was washed extensively with DMF and DCM and solvent removed in a flow of N₂. The peptide was cleaved and deprotected by soaking the resin with a mixture of TFA/TIPS/water/phenol (90/2.5/2.5/5) and shaking at room temperature for 3 hours. After filtration, the filtrate was collected and dried by N₂. The residue was dissolved in ACN and water for HPLC purification using the method: Phenomenex Gemini-NX C18 preparative column (5 um, 110 Å, 50 x 30 mm), 23% ACN, 0.1% TFA in H₂O, isocratic, flow rate 15 mL/min. Retention time for crown-αMSH is 3.7 min. HRMS (ESI) calcd for C₇₂H₁₀₈ N₂₀O₁₆²⁺ [M+2H]²⁺: 798.4383, found: 798.8396

Resultant crown-αMSH construct was labelled with ²²⁵Ac to yield structure (15):

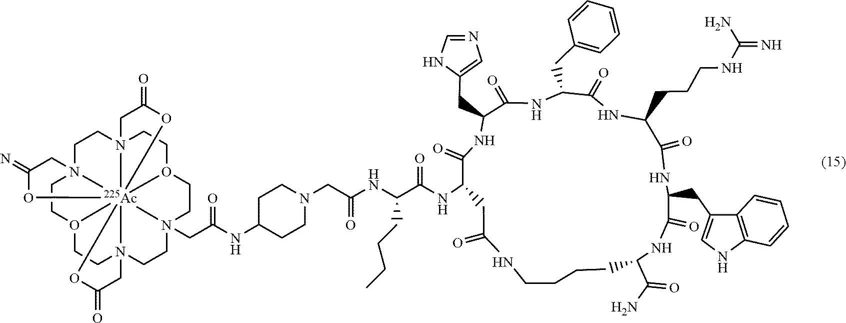

²²⁵Ac was obtained using isotope supplied by Canadian Nuclear Laboratories from the decay of ²²⁹Th which was subsequently separated from aged ²³³U. After purifying by a combination of cation and anion exchange, high purity ²²⁵Ac (>99%) was eluted in concentrated HCl and shipped as an evaporated residue. Upon receipt, the activity was purified again by branched DGA ion-exchange chromatography to remove any potential impurities introduced during acid evaporation.

### Example 4.1 - ²²⁵Ac Labelling of Crown, Crown-TATE and Crown-α-MSH

²²⁵Ac labeling of crown was achieved by mixing the ligand and isotope in acetate buffers between pH 5-7 at ambient temperature. Reactions were monitored by radioTLC. TLC plates were scanned 5 hours after developing to allow the decay of short-lived isotopes, particularly ²¹³Bi that binds stronger with DOTA-type chelators, and for ²²⁵Ac to reach equilibrium. Crown or Crown-TATE (3 µL, 10⁻³ M aqueous unless specified), purified ²²⁵Ac (2.5 uL in 0.05 M HNO₃) and ammonium acetate buffer (20 µL, 1 M, fresh) mixed together and kept at room temperature for 30 minutes.

### Radio TLC Characterization of ²²⁵Ac-Crown-TATE

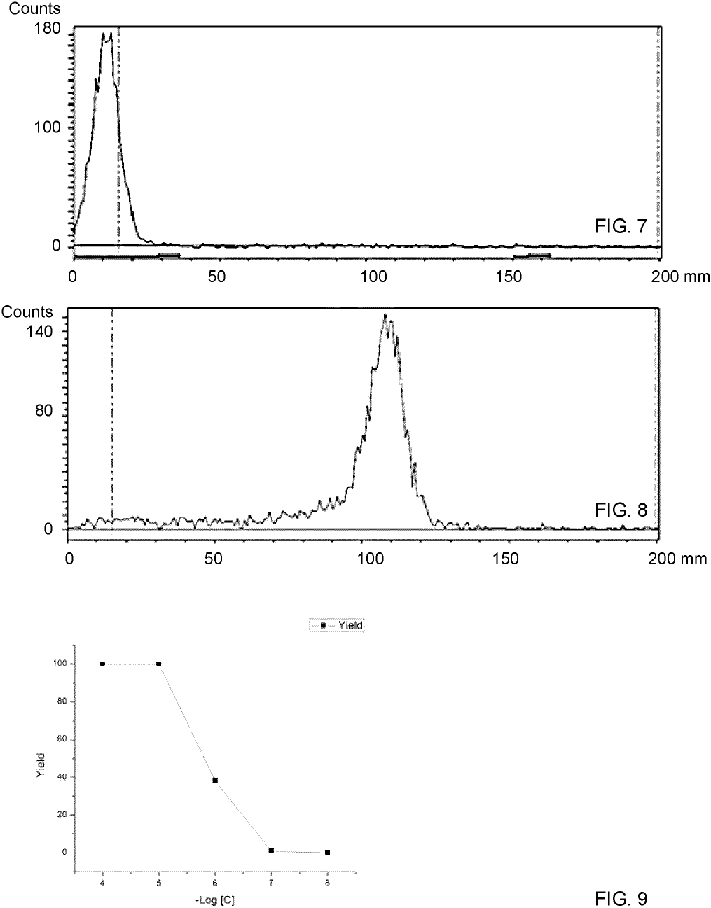

1-5 µL of sample was spotted on SA iTLC plate (silicic acid-embedded instant thin layer chromatography plate) and developed with ethylenediamine tetraacetic acid (EDTA) (pH 5, 0.05 M). Free Ac moves to the solvent front, and Ac labelled products stay close to the bottom of the plate. Alternatively, the TLC can also be developed on a silica plate with Al backing, using citrate buffer (pH 4, 0.4 M). The plate was counted 1 day after to allow the decay of shorter lived isotopes. FIG. 7 shows the results of radioTLC for the Crown-TATE construct with chelated ²²⁵Ac (large peak of labelled ²²⁵Ac-crown-TATE remains at the bottom of the plate), while FIG. 8 shows the comparative results of radioTLC of free ²²⁵Ac (control) (²²⁵Ac moves to the solvent front).

### Labelling Yield at Various Concentrations of Crown and Various pH

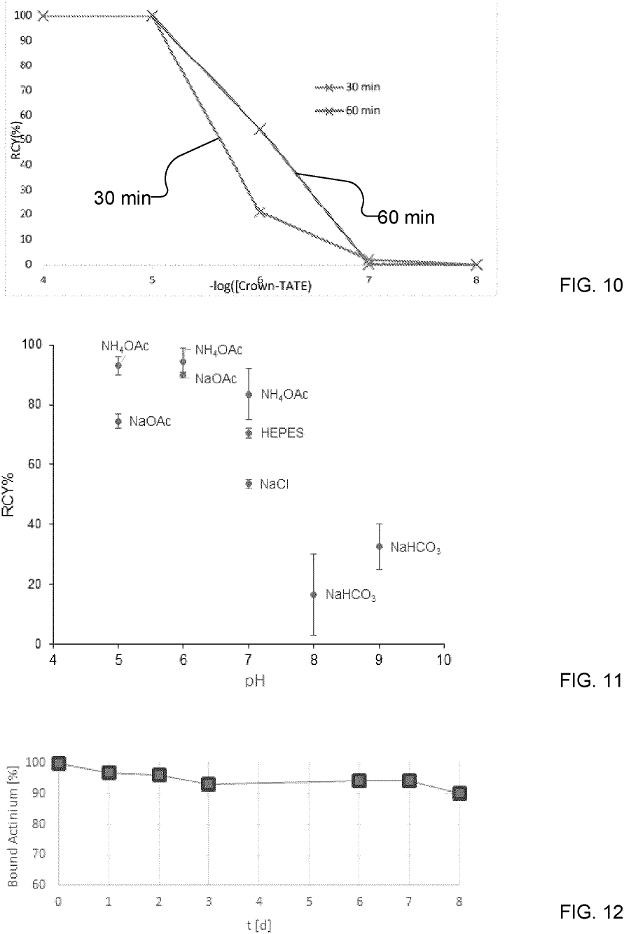

FIG. 9 shows the radiolabelling yield of crown incubated at room temperature for a period of 30 minutes at varying concentrations with purified ²²⁵Ac at pH 7. FIG. 10 shows the radiolabelling yield of crown-TATE incubated at room temperature for a period of 30 minutes or 60 minutes at varying chelator concentrations with purified ²²⁵Ac at pH 7.

FIG. 11 shows the labelling of crown with ²²⁵Ac in various buffers and at various pH levels. Reactions were carried out in various buffer and at various pH levels. Ligand concentration was 10⁻⁶ M and buffer concentration was 0.1 M. pH was adjusted with either NaOH or HNO₃.

Among the buffers studied, NH₄OAc buffers gave the best yield followed by NaOAc. The optimal pH is in the range of about 6 with good yield between pH 5-7, which is a range that convenient to work with. Labelling is also effective at physiological pH, pH 7.4.

The results of this example demonstrate that the crown chelator and the crown-TATE in vivo targeting construct can form a stable complex with the exemplary radiometal ²²⁵Ac at room temperature and neutral pH quantitatively at concentrations as low as 10⁻⁵ M.

### Serum Stability of Crown-TATE in Vivo Targeting Construct

The serum stability of the crown-TATE in vivo targeting construct was evaluated by adding chelated ²²⁵Ac to human serum. The resultant solution was incubated at 37° C. for a period of 8 days and the percentage of actinium that remained bound over this period was monitored. The conditions used for the assay were as follows: 90 µLNH₄OAc buffer: 0.2 M, pH 5.84; 100 µL human serum; 1 uL²²⁵Ac, 50 kBq; crown-TATE: 5x10⁻⁶ M (by adding 10 µL stock); ligand : metal = 10⁴ : 1.

Results are presented in FIG. 12 and Table 2 below (RCY = radiochemical yield, A is of a first reaction A, B is of a second reaction B, SD is standard deviation). The results demonstrate that the tested ²²⁵Ac-crown-TATE construct has good stability in human serum.

TABLE 2

Serum stability of ²²⁵Ac-Crown-TATE

| Time (days) | RCY A (%) | RCY B (%) | SD |
|---|---|---|---|
| 0 | 100 | 100 | 100 |
| 1 | 97.1 | 96.4 | 96.7 |
| 2 | 96.0 | 96.5 | 96.2 |
| 3 | 92.8 | 93.4 | 93.1 |
| 6 | 94.3 | 93.9 | 94.1 |
| 7 | 92.0 | 96.4 | 94.2 |
| 8 | 90.1 | 89.9 | 90.0 |

### Radiolabelling of Crown and Crown-αMSH, Including Compared With DOTA

Stock solutions of crown (10⁻³ M - 10⁻⁶ M) and DOTA (10⁻³ M - 10⁻⁶ M) were prepared in water. Ligand (1 µL), buffer (1 M pH 7 NH₄OH, 1 µL), water (7 µL), and ²²⁵Ac stock (1 µL, 10-30 kBq) were mixed together. For crown, the reaction was kept at room temperature for 15 min and for DOTA, the reaction was at 90° C. for 15 min. 2-5 µL was taken for radioTLC monitoring at various time points. The sample was spotted on a silica plate with aluminium backing (10 cm x 1.5 cm) and developed with sodium citrate (0.4 M, pH 4). The plates were scanned >5 hours later when all the daughters reach secular equilibrium. Under those conditions, unchelated actinium migrates to the solvent front (Rf=1) and chelated actinium remains close baseline (R_{f}<0.5). n=3 for each data point.

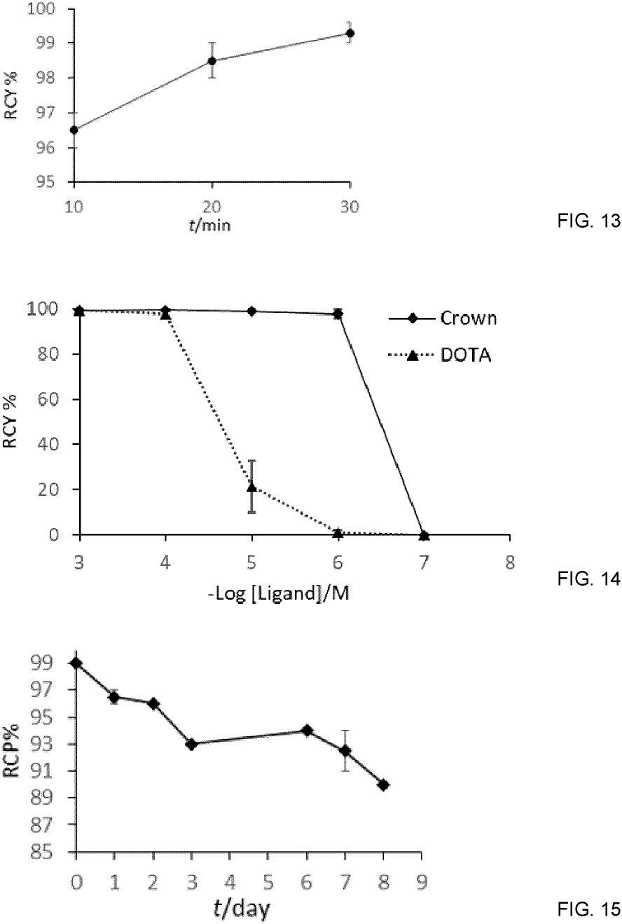

As shown in FIG. 13 for the chelation of ²²⁵Ac by crown, the reaction at crown concentration of 10⁻⁵ M was monitored by radioTLC at 10, 20 and 30 min. The reaction reached >96% at 10 min.

When comparing the labelling of crown vs. DOTA, quantitative labeling was achieved at crown concentrations >10⁻⁶ M, or DOTA >10⁻⁴ M (FIG. 14). The reaction was largely complete after 10 min. When left at ambient temperature, <2% free ²²⁵Ac was observed for up to 5 days and up to 8 days in a second experiment, indicating prolonged complex stability. To obtain the data shown in FIG. 14, labeling with crown was conducted at room temperature for a period of 15 minutes, while labelling with DOTA was conducted at 90° C. for a period of 15 minutes.

Serum stability of the ²²⁵Ac-crown-αMSH was examined. Crown-αMSH stock (10 µL, 10⁻⁴ M), NHaOAc buffer (90 µL, 0.2 M, pH 7) and 1 µL ²²⁵Ac (15 kBq) was mixed together and kept at room temperature for 35 min. After adding 100 µL human serum, the solution was kept at 37° C. for 8 days. Radiochemical purity was monitored periodically by radioTLC. The sample was spotted on an iTLC-SA strip (12 cm x 1.5 cm) and developed with EDTA (0.05 M, pH 5). The plates were scanned >5 hours later when all the daughters reach secular equilibrium. Under those conditions, unchelated actinium migrates to the solvent front (R_{f}=1) and chelated actinium remains at baseline (R_{f}=0). N=3 for each data point. Results are shown in FIG. 15 (RCP = radiochemical purity).

### Labelling of Crown, Crown-TATE and Crown-αMSH With Other Radiometals

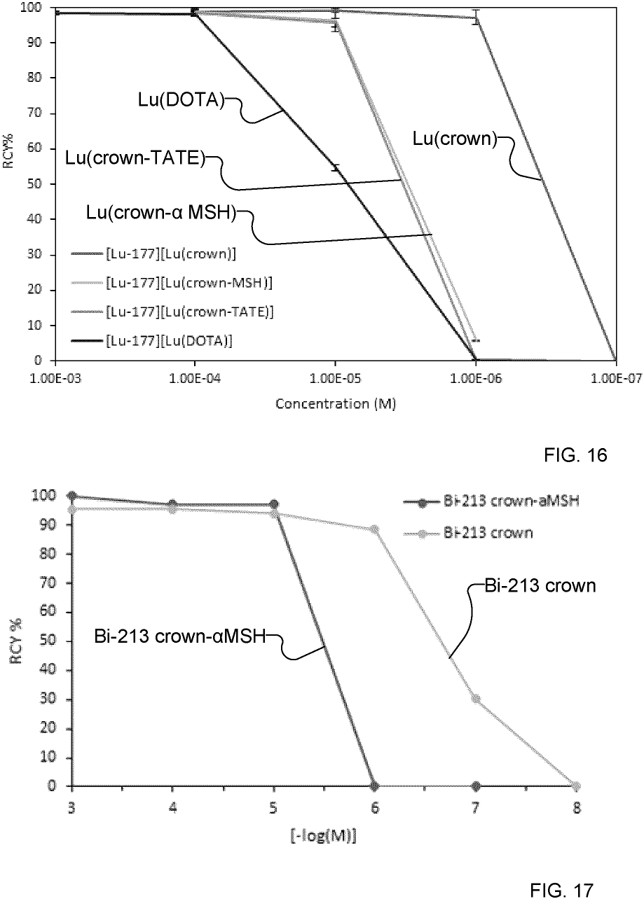

Experiments were also conducted to evaluate the labelling of crown, crown-αMSH and crown-TATE with other radiometals. Results for the three constructs and a comparative chelator DOTA chelating ¹⁷⁷Lu are shown in FIG. 16 at various concentrations of chelator. Crown showed significantly improved labelling with ¹⁷⁷Lu over DOTA, labelling could be conducted at room temperature in 15 minutes for crown versus DOTA required a temperature of 90° C. for a period of 30 minutes. Also for crown, quantitative labelling was achieved at 10⁻⁶M versus 10⁻⁴M for DOTA.

FIG. 17 shows labelling of crown and crown-αMSH at varying concentrations of chelator with ²¹³Bi. The reaction was carried out at room temperature for a period of 8 minutes in MES buffer (2-(N-morpholino)ethanesulfonic acid).

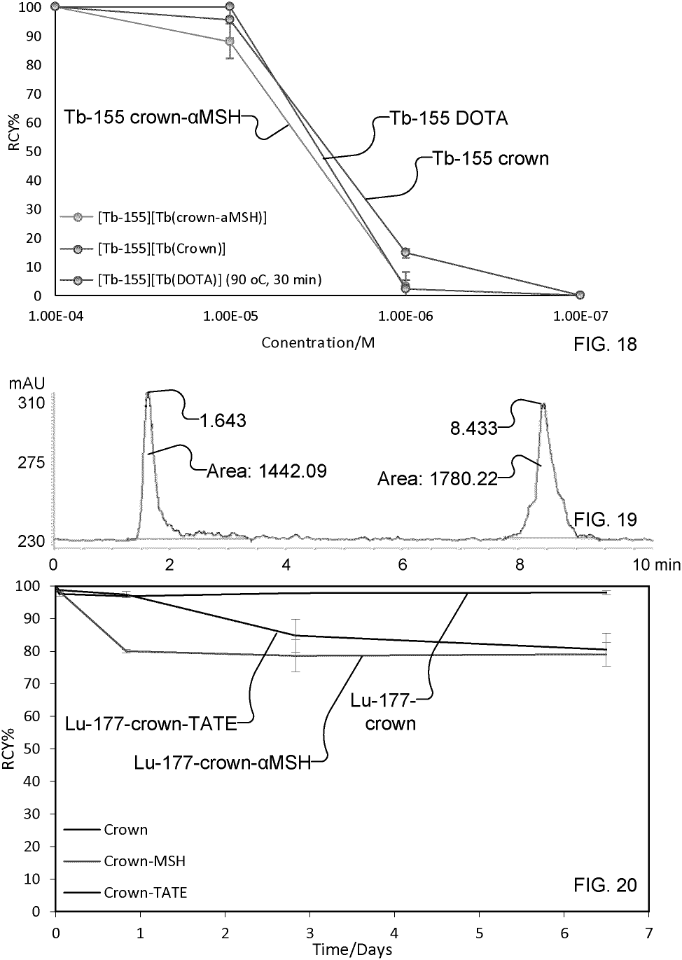

FIG. 18 shows labelling of crown and crown-αMSH at varying concentrations of chelator with ¹⁵⁵ Tb. The reaction was carried out at room temperature for a period of 15 minutes in ammonium acetate buffer. Data for ¹⁵⁵ Tb labelling of DOTA is also included, although labelling of DOTA was conducted at 90° C. for a period of 30 minutes.

Labelling of crown-TATE with ⁶⁸Ga was also evaluated. The tested reaction included 2 nmol of crown-TATE with approximately 4 MBq ⁶⁸Ga in ethyl acetate buffer. The pH was approximately 4-5 and the reaction was conducted at room temperature for 15 minutes. Radiochemical conversion under these conditions was determined to be 55% by HPLC (results shown in FIG. 19).

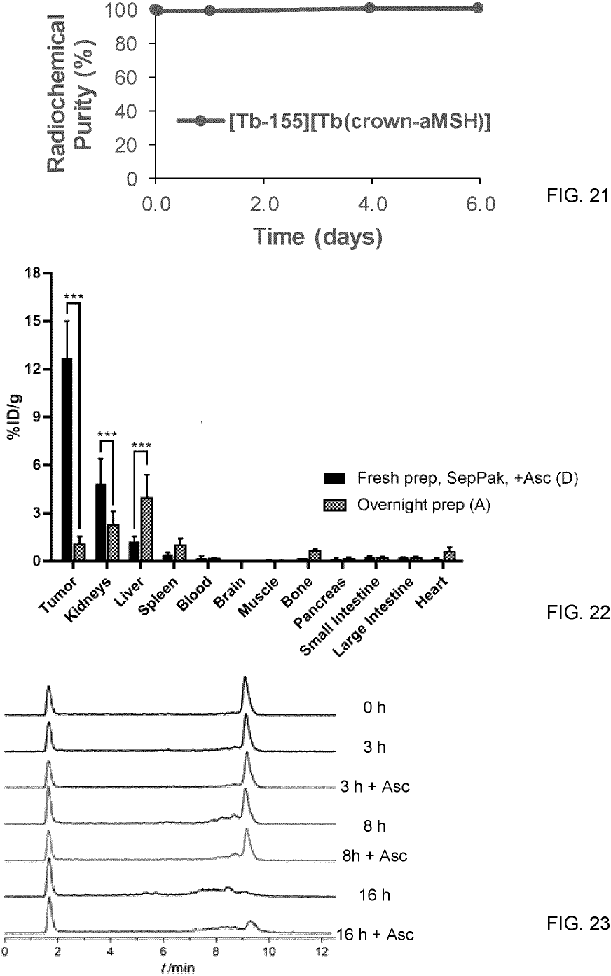

FIG. 20 shows the serum stability of crown, crown-TATE and crown-αMSH at 37° C. labelled with ¹⁷⁷Lu, and FIG. 21 shows the serum stability of crown-αMSH at 37° C. labelled with ¹⁵⁵Tb.

αMSH was modified with crown using one of the four pendant carboxylic groups as a linker to generate an overall neutrally charged complex upon binding with 3+ metals. Since no need for a bifunctional ligand is required, this approach represents an easy synthesis of a conjugation ready ligand (crown-3tBu). The stability of an αMSH targeting moiety with an amide linker was previously demonstrated.^{[38]}

In a similar method as described above, ²²⁵Ac -crown-αMSH was generated upon incubation of the modified peptide with ²²⁵Ac in acetate buffer. High radiolabeling yields (> 98%) were achieved at levels up to 6 MBq. When purification was required, light C18 Sep-Pak™ was used to remove free ²²⁵Ac. Molar activity for the final product was determined to be 4.1 ± 1.9 MBq/nmol (n = 5), the highest among reported methods to date.^{[20,22]} Serum stability study indicated ~90% ²²⁵Ac remain chelated over 8 days.

### Example 5.1 - Biological Distribution of ²²⁵Ac-Crown-αMSH

The inventors performed an in vivo evaluation to establish the biodistribution profile of ²²⁵Ac-crown-αMSH in B16F10 tumor bearing mice.

### Cell Culture

The B16F10 cell line (Mus musculus) was obtained commercially from ATCC (CRL-6475), and confirmed pathogen-free using the IMPACT 1 mouse profile (IDEXX BioResearch). The cells were cultured in DMEM media (StemCell Technologies) supplemented with 10% FBS, 100 U/mL penicillin and 100 µg/mL streptomycin at 37° C. in a humidified incubator containing 5% CO₂.

### Tumor Inoculation

All animal experiments were conducted according to the guidelines established by Canadian Council on Animal Care and approved by Animal Ethics Committee of the University of British Columbia (UBC). Male C57BL/6J mice were acquired in-house and kept under pathogen-free conditions in the Animal Resource Centre at the BC Cancer Research Centre. The mice were anesthetised by inhalation of 2% isoflurane in 2 L/min oxygen, and 1 x10⁶ B16F10 cells were inoculated subcutaneously at right flank. Two to four days after inoculation, the mice were transferred to the UBC Centre of Comparative Medicine, where biodistribution studies were performed once the tumors reached 8-10 mm.

### Biodistribution Study

At the day of biodistribution mice were injected in the tail vein with ~20 kBq of ²²⁵Accrown-αMSH (range: 10.8-31.6 kBq). After injection mice were allowed to roam freely in their cages, and they were euthanized in groups of 4 at 2 hours post injection by CO₂ asphyxiation under isoflurane anesthesia. Blood was collected by cardiac puncture and a full biodistribution performed. Organs were cleaned from blood, weighed and the activity determined using a calibrated gamma counter (Packard Cobra II Auto-gamma counter, Perkin Elmer, Waltham, MA, USA) using three energy windows: 60-120 keV (window A), 180-260 keV (window B), and 400-480 keV (window C). Counting was performed after 6 hours post-sacrifice to ensure equilibrium of the ²²⁵Ac decay chain. Counts were decay corrected from the time of sacrifice and total organ weights were used for the calculation of injected dose per gram of tissue (% ID/g). No differences were noted between the data calculated by three different windows; therefore, the biodistributions are reported using the data acquired using window A.

### Results

The inventors evaluated biodistribution of ²²⁵Ac-crown-αMSH in mice bearing B16F10 tumors at 2 h post-injection (p.i.) in two separate studies using a total of 5 groups of mice (Table 3). In the first study, three conditions were evaluated: ²²⁵Ac-crown-αMSH prepared the day before (18 h) (A, FIG. 22, right bars), same day (4 h) (B), or same day (4 h) with Sep-Pak purification (C); in the second study the inventors compared 225Ac-crown-αMSH labelled 4 hours before with Sep-Pak purification and with the introduction of 0.1 M L-ascorbate (D, FIG. 22, left bars), or ²²⁵Ac-crown-αMSH prepared 18 hours prior with L-ascorbate (E).

The construct prepared the previous day demonstrated poor tumor uptake, while freshly synthesized compound gave excellent tumor to background ratios (A vs. B). Sep-Pak purification improved tumor-to-background ratios, but results were not statistically significant (B vs. C, adjusted P value 0.06-0.61). With the addition of L-ascorbate, the inventors observed higher tumor uptake but tumor-to-background ratios are not statistically different (C vs. D, adjusted P value 0.11-0.89). Despite containing 0.1 M L-ascorbate, a sample left overnight did not contain viable radiopharmaceutical upon injection (E). High energy ionized helium (5-8.5 MeV) generated by ²²⁵Ac can cause extensive radiolysis of water and generate a range of reactive oxygen species.^{[39]} It is therefore not surprising that biomolecules degrade as a result. The small difference between Sep-Pak purified and unpurified samples could be attributed to the presence of EtOH used for elution acting as a ROS scavenger.

It is important to note that in all 5 cases, radioTLC showed > 98% radiochemical purity. TLC is a widely used method for the rapid determination of radiopharmaceutical purity prior to injection. In addition, current IAEA guidelines on quality control for ²²⁵Ac radiopharmaceuticals list only radioTLC as the method of choice for rapidly assessing compound integrity.^{[40]} For the purpose of this study, the inventors investigated using radioHPLC as an additional quality control method. The gamma signal from ²²⁵Ac is mostly from ²²¹Fr (11.4%, 218 keV), ²¹³Bi (25.9%, 440 keV) and Compton peaks, not from ²²⁵Ac itself. Therefore, peak areas on gamma traces do not necessarily reflect the radiochemical purity of ²²⁵Ac-crown-αMSH. The large solvent front peak (T_{R} ~1.7 min, FIG. 23) is mostly due to ²¹²Fr, as determined by collecting this peak and analyzing by gamma spectroscopy. In contrast, ²¹³Bi exhibits similar chelation chemistry to Ac and can be found bound to the crown-peptide (T_{R} ~9.4 min, FIG. 23). As shown in FIG. 23, the peptide peak slowly degraded overtime. Addition of L-ascorbate (0.1 M) slowed degradation, but not enough to maintain sufficient compound activity the following day. Therefore it is desirable to reduce the time between the end of synthesis and injection. For quality control, it is recommended to use radioTLC and gamma spectroscopy (cut and count ²²¹Fr after 30 min) to assess the presence of free ²²⁵Ac and use HPLC to assess the integrity of the peptide. Another common practice is to add DTPA as a chelator for residual free metal ions.^{[40]} Upon addition of 1000 eq. of DTPA to ²²⁵Ac-crown-αMSH, the inventors observed slow transmetallation on HPLC (64% in 7 hours).

TABLE 3

Biodistribution of ²²⁵Ac-crown-αMSH in mice bearing B16F10 tumors at 2 hours post-injection (results reported in %lD/g, with mean±standard deviation) with various preparation. Ratios of the biodistribution of ²²⁵Ac-crown-αMSH in tumor versus blood, muscle, bone, kidney and liver are presented at the bottom.

| | A | | B | | C | | D* | | E | |
|---|---|---|---|---|---|---|---|---|---|---|
| Fresh | N | | Y | | Y | | Y | | N | |
| SPK | N | | N | | Y | | Y | | N | |
| L-Asc | N | | N | | N | | Y | | Y | |
| Tissue (n=4) | Avg | SD | Avg | SD | Avg | SD | Avg | SD | Avg | SD |
| Blood | 0.15 | 0.04 | 0.05 | 0.02 | 0.06 | 0.02 | 0.20 | 0.13 | 0.31 | 0.11 |
| Urine | 143.72 | 99.45 | 27.18 | 13.86 | 19.16 | 13.18 | 46.00 | 46.43 | 81.21 | 75.98 |
| Feces | 1.00* | 1.36* | 0.31 | 0.24 | 0.21 | 0.21 | 0.25 | 0.17 | 0.55 | 0.40 |
| Tumour | 1.06 | 0.50 | 4.58 | 2.28 | 6.62 | 1.51 | 12.70 | 2.31 | 4.84 | 3.20 |
| Brain | 0.02 | 0.00 | 0.01* | 0.00* | 0.01 | 0.01 | 0.03 | 0.00 | 0.09 | 0.06 |
| Muscle | 0.03 | 0.02 | 0.06 | 0.05 | 0.02 | 0.02 | 0.05 | 0.01 | 0.18 | 0.08 |
| Bone | 0.62 | 0.15 | 0.16 | 0.06 | 0.17 | 0.04 | 0.20 | 0.00 | 1.82 | 0.38 |
| Bladder | 7.85 | 5.30 | 2.36 | 1.06 | 1.04 | 1.00 | 2.09 | 1.86 | 3.44 | 2.34 |
| Spleen | 1.00 | 0.44 | 0.30 | 0.06 | 0.15 | 0.08 | 0.43 | 0.11 | 2.75 | 1.00 |
| Pancreas | 0.14 | 0.12 | 0.11 | 0.14 | 0.09 | 0.12 | 0.14 | 0.08 | 0.22 | 0.11 |
| Stomach | 0.21* | 0.07* | 0.34 | 0.07 | 0.36 | 0.13 | 1.04 | 0.36 | 1.18 | 0.15 |
| Sm Intestine | 0.23 | 0.08 | 0.13 | 0.05 | 0.12 | 0.03 | 0.28 | 0.06 | 0.68 | 0.05 |
| Lg Intestine | 0.22 | 0.08 | 0.16 | 0.08 | 0.13 | 0.05 | 0.23 | 0.04 | 0.67 | 0.20 |
| Kidneys | 2.25 | 0.88 | 1.62 | 0.59 | 2.03 | 0.53 | 4.86 | 1.55 | 4.23 | 0.99 |
| Liver | 3.95 | 1.46 | 0.75 | 0.20 | 0.70 | 0.22 | 1.24 | 0.32 | 15.38 | 2.08 |
| Heart | 0.58 | 0.29 | 0.13 | 0.05 | 0.12 | 0.05 | 0.16 | 0.02 | 1.39 | 1.48 |
| Lungs | 0.97 | 0.74 | 0.36 | 0.25 | 0.12 | 0.06 | 0.32 | 0.01 | 1.33 | 0.58 |
| T/Blood | 7.41 | 4.47 | 108.38 | 97.94 | 113.66 | 28.35 | 85.37 | 46.81 | 16.46 | 9.05 |
| T/Muscle | 56.76 | 31.36 | 108.77 | 61.19 | 295.49 | 64.66 | 280.93 | 128.93 | 26.01 | 9.77 |
| T/Bone | 1.70 | 0.58 | 33.58 | 20.58 | 39.44 | 6.79 | 63.51 | 13.16 | 2.54 | 1.29 |
| T/Kidney | 0.50 | 0.20 | 2.82 | 0.71 | 3.28 | 0.13 | 2.80 | 0.98 | 1.08 | 0.49 |
| T/Liver | 0.28 | 0.13 | 6.00 | 1.94 | 9.83 | 1.91 | 10.55 | 2.34 | 0.31 | 0.18 |

*n=3, one mouse with tumor size too small (0.0622 g).

A closer examination of ²²⁵Ac-crown-αMSH biodistribution data at 2 hours post-injection (p.i.) in mice bearing B16F10 melanoma (D, FIG. 22 and Table 3), showed a similar uptake profile to [⁶⁸Ga]Ga-DOTA-αMSH (CCZ01048)^{[29]} with extremely low normal tissue accumulation. In comparing with ²²⁵Ac-DOTA-αMSH,^{[22]} the crown complex showed much higher tumor uptake at 12.7±2.31 %ID/g, and superior tumor-to-normal tissue uptake ratios. For ²²⁵Ac-DOTA-αMSH, tumor to blood, bone and kidneys ratios were 20.4±3.4, 23.2±10.3 and 1.1±0.1 respectively,^{[22]} and for ²²⁵Ac-crown-αMSH, those ratios were 85.4±46.8, 63.5±13.2 and 2.80±0.98 respectively (D). This is likely due to the much higher molar activity of ²²⁵Ac-crown-αMSH, i.e. 1.6 kBq/nmol (DOTA) vs. 4.1 MBq/nmol (crown). Other than the clearance track (kidneys, bladder and urine), the uptake in normal tissue was extremely low for this complex, which gave excellent tumor-to-background ratios (Table 3, C&D). This coupled with the fast internalization capability of the peptide, demonstrates good potential for use of crown as a chelator in in vivo diagnostic and therapeutic applications. Further, the data described herein is directly comparable to the data obtained by Ramogida et al.^{[22]} for the ²²⁵Ac-DOTA-αMSH construct since the targeting moiety and the animal model used were the same across both studies, demonstrating the superiority of crown as a chelator over the current gold standard DOTA, yielding a specific activity that is approximately 100 times greater.

The foregoing experiments demonstrate that crown is capable of incorporating ²²⁵Ac at ambient temperature and high molar activity. When incorporated into a biological targeting construct as ²²⁵Ac-crown-αMSH, the targeting construct could be used to target MC1R expressed in melanoma tumors. In vivo evaluation in mice bearing B16F10 melanoma tumors showed excellent target-to-normal tissue ratios. During the development of this radiopharmaceutical, the inventors discovered the deficiency of current radioTLC based quality control methodology for determination of ²²⁵Ac compound integrity. The inventors recommend the use of HPLC to confirm compound purity, and suggest use of ²²⁵Ac-labeled radiopharmaceuticals with a short delay between production and injection in order to minimize degradation by radiolysis.

### Example 5.2 - Biological Distribution of ²²⁵Ac-Crown-TATE

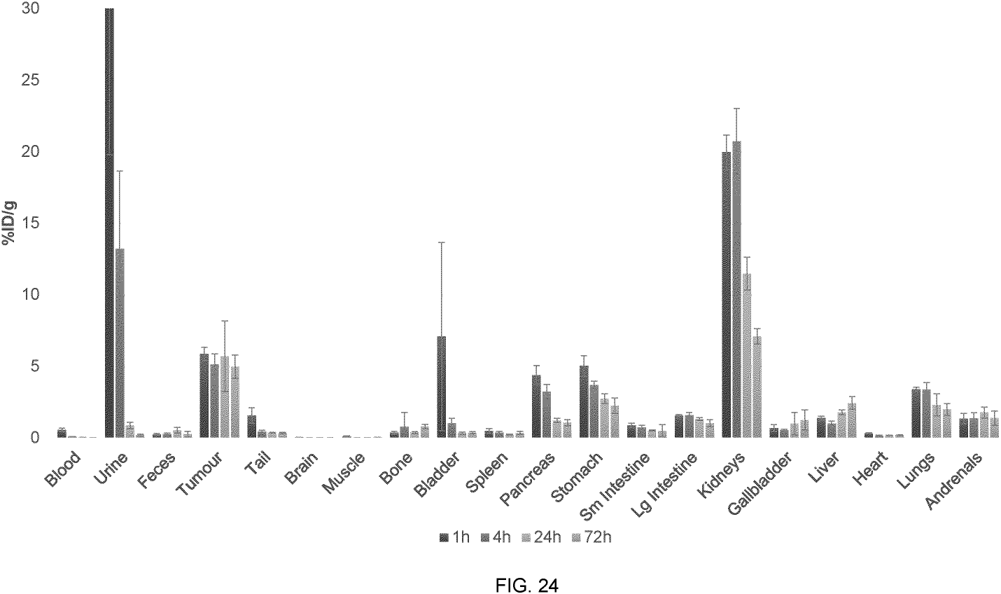

The biological distribution of ²²⁵Ac-crown-TATE was examined by the inventors following similar protocols as described above for Example 5.1. Results are shown in FIG. 24 and Table 4.

TABLE 4

Biodistribution of ²²⁵Ac-crown-TATE in mice bearing B16F10 tumors at indicated time post-injection (results reported in %ID/g, with mean±standard deviation). Ratios of the biodistribution of ²²⁵Ac-crown-TATE in tumor versus blood, muscle, bone, kidney and liver are presented at the bottom.

| | 1 hour | | 4 hours | | 24 hours | | 72 hours | |
|---|---|---|---|---|---|---|---|---|
| | Average | SD | Average | SD | Average | SD | Average | SD |
| Blood | 0.54 | 0.13 | 0.09 | 0.02 | 0.03 | 0.00 | 0.02 | 0.01 |
| Urine | 107.61 | 87.84 | 13.21 | 5.44 | 0.87 | 0.22 | 0.20 | 0.07 |
| Feces | 0.22 | 0.09 | 0.27 | 0.06 | 0.53 | 0.20 | 0.26 | 0.20 |
| Tumour | 5.86 | 0.48 | 5.15 | 0.73 | 5.70 | 2.46 | 4.97 | 0.82 |
| Tail | 1.56 | 0.55 | 0.42 | 0.11 | 0.35 | 0.02 | 0.34 | 0.06 |
| Brain | 0.03 | 0.00 | 0.01 | 0.00 | 0.01 | 0.00 | 0.01 | 0.00 |
| Muscle | 0.10 | 0.04 | 0.02 | 0.00 | 0.02 | 0.00 | 0.04 | 0.01 |
| Bone | 0.35 | 0.12 | 0.78 | 1.00 | 0.38 | 0.06 | 0.78 | 0.15 |
| Bladder | 7.09 | 6.57 | 1.04 | 0.32 | 0.33 | 0.08 | 0.37 | 0.07 |
| Spleen | 0.48 | 0.17 | 0.36 | 0.11 | 0.23 | 0.03 | 0.35 | 0.11 |
| Pancreas | 4.39 | 0.67 | 3.24 | 0.52 | 1.22 | 0.15 | 1.07 | 0.21 |
| Stomach | 5.04 | 0.71 | 3.70 | 0.28 | 2.75 | 0.33 | 2.25 | 0.55 |
| Sm Intestine | 0.88 | 0.13 | 0.74 | 0.15 | 0.51 | 0.04 | 0.49 | 0.43 |
| Lg Intestine | 1.55 | 0.08 | 1.58 | 0.18 | 1.31 | 0.11 | 1.02 | 0.26 |
| Kidneys | 19.97 | 1.20 | 20.73 | 2.30 | 11.48 | 1.14 | 7.09 | 0.54 |
| Gallbladder | 0.68 | 0.24 | 0.52 | 0.09 | 0.99 | 0.79 | 1.25 | 0.70 |
| Liver | 1.38 | 0.14 | 1.00 | 0.15 | 1.77 | 0.17 | 2.43 | 0.45 |
| Heart | 0.32 | 0.03 | 0.15 | 0.03 | 0.18 | 0.02 | 0.19 | 0.03 |
| Lungs | 3.40 | 0.16 | 3.37 | 0.48 | 2.31 | 0.78 | 2.00 | 0.41 |
| Andrenals | 1.34 | 0.37 | 1.37 | 0.38 | 1.77 | 0.38 | 1.37 | 0.50 |
| T/Blood | 11.57 | 3.53 | 59.97 | 14.94 | 162.29 | 62.84 | 402.51 | 173.92 |
| T/Muscle | 63.37 | 18.19 | 255.70 | 70.90 | 334.05 | 106.00 | 135.35 | 24.52 |
| T/Bone | 17.85 | 4.46 | 21.65 | 11.71 | 14.49 | 3.64 | 6.40 | 0.45 |
| T/Kidney | 0.30 | 0.04 | 0.25 | 0.04 | 0.48 | 0.15 | 0.70 | 0.09 |
| T/Liver | 4.31 | 0.67 | 5.31 | 1.19 | 3.18 | 1.19 | 2.06 | 0.17 |

### Example 5.3 - Biological Distribution of ²¹³Bi-Crown-αMSH

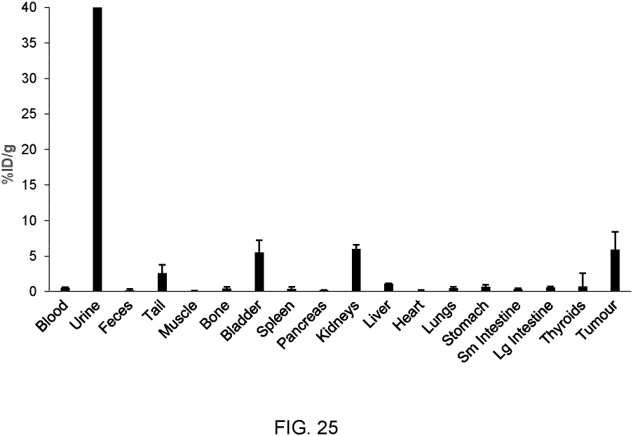

A similar study to Example 5.1 was conducted using ²¹³Bi instead of ²²⁵Ac. Biodistribution was evaluated at 1 hour post injection, yielding the results in shown in Table 5 and FIG. 25. Similar results as for ²²⁵Ac were observed, i.e. the radiometal showed good accumulation in tumour tissue and not a significant accumulation in other tissues.

TABLE 5

Biodistribution of ²¹³Bi-crown-αMSH in mice bearing B16F10 tumors at 1 hour post-injection (results reported in percent of injected dose per tram (%ID/g), with mean±standard deviation). Ratios of the biodistribution of ²¹³Bi-crown-αMSH in tumor versus blood, muscle, bone, kidney and liver are presented at the bottom.

| Tissue | Average (%ID/g) | SD |
|---|---|---|
| Blood | 0.50 | 0.12 |
| Urine | 93.18 | 51.10 |
| Feces | 0.23 | 0.18 |
| Tail | 2.56 | 1.22 |
| Muscle | 0.08 | 0.03 |
| Bone | 0.41 | 0.24 |
| Bladder | 5.48 | 1.74 |
| Spleen | 0.36 | 0.34 |
| Pancreas | 0.15 | 0.05 |
| Kidneys | 6.02 | 0.58 |
| Liver | 1.09 | 0.06 |
| Heart | 0.09 | 0.12 |
| Lungs | 0.45 | 0.23 |
| Stomach | 0.67 | 0.30 |
| Sm Intestine | 0.34 | 0.11 |
| Lg Intestine | 0.56 | 0.16 |
| Thyroids | 0.74 | 1.84 |
| Tumour | 5.90 | 2.50 |
| T/Blood | 12.15 | 5.63 |
| T/Muscle | 84.48 | 46.40 |
| T/Bone | 23.41 | 20.69 |
| T/Kidney | 0.99 | 0.41 |
| T/Liver | 5.44 | 2.28 |

### Example 6.0 - Methods for ²²⁵Ac Measurement and Quantification

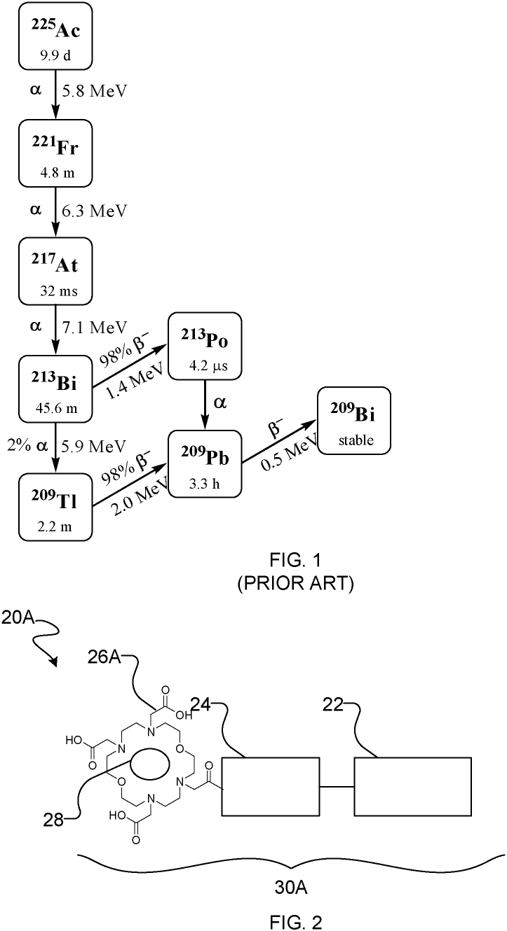

²²⁵Ac has seven daughter isotopes (FIG. 1). Among them, ²²¹Fr and ²¹³Bi have distinct gamma emissions that can be used to quantify ²²⁵Ac. ²²¹Fr reaches 99% of ²²⁵Ac in 32 min, and ²¹³Bi reaches 99% of ²²⁵Ac in 292 min (4.86 h). When gamma spectroscopy was necessary, the inventors waited for >30 min to use ²²¹Fr (218 keV) to quantify ²²⁵Ac, or waited for >5 h to use both ²²¹Fr and ²¹³Bi (440 keV) to quantify ²²⁵Ac. When radioTLC is required, waiting for >5 h to scan the plate is necessary to decay ²¹³Bi, which often binds stronger with the ligands. Alternatively, the inventors cut the TLC plates and counted with gamma spectroscopy after 30 min. Similarly, for biodistribution studies, the tissues collected were counted for >5 h. Reading at different energy windows (60-120 keV, 180-260 keV, 400-480 keV) produced the same results, with 60-120 keV giving the highest counts. For radio-HPLC, the gamma detector was set to the full range 19-1100 keV to maximize the signal, which mostly came from ²²¹Fr and ²¹³Bi. Therefore, radio-HPLC gamma trace is not a quantitative reflection of the labeling yield. The fractions need to be collected and counted in gamma spectroscopy to generate such information when needed.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

### References

The following references are of interest with respect to the subject matter herein, and are hereby incorporated by reference herein in their entireties:
S. Gudkov, N. Shilyagina, V. Vodeneev, A. Zvyagin, Int. J. Mol. Sci. 2015, 17, 33.
J. P. Pouget, I. Navarro-Teulon, M. Bardiés, N. Chouin, G. Cartron, A. Pélegrin, D. Azria, Nat. Rev. Clin. Oncol. 2011, 8, 720-734.
J. Strosberg, G. El-Haddad, E. Wolin, A. Hendifar, J. Yao, B. Chasen, E. Mittra, P. L. Kunz, M. H. Kulke, H. Jacene, et al., N. Engl. J. Med. 2017, 376, 125-135.
B. Sangro, L. Carpanese, R. Cianni, R. Golfieri, D. Gasparini, S. Ezziddin, P. M. Paprottka, F. Fiore, M. Van Buskirk, J. Ignacio Bilbao, et al., Hepatology 2011, 54, 868-878.
S. D. Bos, Prostate 1994, 25, 23-26.
O. Sartor, Rev. Urol. 2004, Suppl 10, S3-S12.
C. Parker, S. Nilsson D Heinrich, S. I. Helle, J. M. O’Sullivan, S. D. Fossa, A. Chodacki, P. Wiechno, J. Logue, M. Seke, A. Widmark, et al., N. Engl. J. Med. 2013, 369, 213-223.
A. Morgenstern, C. Apostolidis, C. Kratochwil, M. Sathekge, L. Krolicki, F. Bruchertseifer, Curr. Radiopharm. 2018, 11, 200-208.
D. A. Scheinberg, M. R. McDevitt, Curr. Radiopharm. 2012, 4, 306-320.
G. Sgouros, J. C. Roeske, M. R. McDevitt, S. Palm, B. J. Allen, D. R. Fisher, A. B. Brill, H. Song, R. W. Howell, G. Akabani, et al., J. Nucl. Med. 2010, 51, 311-328.
C. Wulbrand, C. Seidl, F. C. Gaertner, F. Bruchertseifer, A. Morgenstern, M. Essler, R. Senekowitsch-Schmidtke, PLoS One 2013, 8, DOI 10.1371 /journal. pone.0064730.
C. Kratochwil, F. Bruchertseifer, F. L. Giesel, M. Weis, F. A. Verburg, F. Mottaghy, K. Kopka, C. Apostolidis, U. Haberkorn, A. Morgenstern, J. Nucl. Med. 2016, 57, 1941-1944.
C. Kratochwil, F. Bruchertseifer, H. Rathke, M. Hohenfellner, F. L. Giesel, U. Haberkorn, A. Morgenstern, J. Nucl. Med. 2018, 59, 795-802.
A. K. H. Robertson, C. F. Ramogida, P. Schaffer, V. Radchenko, Curr. Radiopharm. 2018, 11, 156-172.
S. L. Wu, W. DeW. Horrocks, J. Chem. Soc. Dalt. Trans. 1997, 1497-1502.
M. R. McDevitt, D. Ma, J. Simon, R. K. Frank, D. A. Scheinberg, Appl. Radiat. Isot. 2002, 57, 841-847.
M. Miederer, G. Henriksen, A. Alke, I. Mossbrugger, L. Quintanilla-Martinez, R. Senekowitsch-Schmidtke, M. Essler, Clin. CancerRes. 2008, 14, 3555-3561.
M. R. McDevitt, D. Ma, L. T. Lai, J. Simon, P. Borchardt, R. K. Frank, K. Wu, V. Pellegrini, M. J. Curcio, M. Miederer, et al., Science 2001, 294, 1537-1540.
N. A. Thiele, V. Brown, J. M. Kelly, A. Amor-Coarasa, U. Jermilova, S. N. MacMillan, A. Nikolopoulou, S. Ponnala, C. F. Ramogida, A. K. H. Robertson, et al., Angew. Chem. Int. Ed. 2017, 56, 14712-14717.
J. M. Kelly, A. Amor-Coarasa, S. Ponnala, A. Nikolopoulou, C. Williams, N. A. Thiele, D. Schlyer, J. J. Wilson, S. G. DiMagno, J. W. Babich, J. Nucl. Med. 2019, 60, 649-655.
P. Comba, U. Jermilova, C. Orvig, B. O. Patrick, C. F. Ramogida, K. Rück, C. Schneider, M. Starke, Chem. - A Eur. J. 2017, 23, 15945-15956.
C. F. Ramogida, A. K. H. Robertson, U. Jermilova, C. Zhang, H. Yang, P. Kunz, J. Lassen, I. Bratanovic, V. Brown, L. Southcott, et al., EJNMMIRadiopharm. Chem. 2019, 4, 1-20.
B. W. Stein, A. Morgenstern, E. R. Batista, E. R. Birnbaum, S. E. Bone, S. K. Cary, M. G. Ferrier, K. D. John, J. L. Pacheco, S. A. Kozimor, et al., J. Am. Chem. Soc. 2019, 141, 19404-19414.
S. J. Kennel, L. L. Chappell, K. Dadachova, M. W. Brechbiel, T. K. Lankford, I. A. Davis, M. Stabin, S. Mirzadeh, Cancer Biother. Radiopharm. 2000, 15, 235-244.
S. Ray Banerjee, M. Pullambhatla, C. A. Foss, A. Falk, Y. Byun, S. Nimmagadda, R. C. Mease, M. G. Pomper, J. Med. Chem. 2013, 56, 6108-6121.
B. Mitran, Z. Varasteh, R. K. Selvaraju, G. Lindeberg, J. Sörensen, M. Larhed, V. Tolmachev, U. Rosenström, A. Orlova, Int. J. Oncol. 2016, 48, 2124-2134.
A. Barge, L. Tei, D. Upadhyaya, F. Fedeli, L. Beltrami, R. Stefania, S. Aime, G. Cravotto, Org. Biomol. Chem. 2008, 6, 1176-1184.
S.-L. Ma, W.-X. Zhu, S.-J. Dong, Q.-L. Guo, Y.-B. She, Chinese J. Chem. 2010, 21, 1178-1182.
C. Zhang, Z. Zhang, K. S. Lin, J. Pan, I. Dude, N. Hundal-Jabal, N. Colpo, F. Benard, Theranostics 2017, 7, 805-813.
M. A. Postow, M. K. Callahan, J. D. Wolchok, J. Clin. Oncol. 2015, 33, 1974-82.
C. Hertzman Johansson, S. Egyhazi Brage, Pharmacol. Ther 2014, 142, 176-182.
C. S. Hinrichs, S. A. Rosenberg, Immunol. Rev. 2014, 257, 56-71.
R. H. I. Andtbacka, H. L. Kaufman, F. Collichio, T. Amatruda, N. Senzer, J. Chesney, K. A. Delman, L. E. Spitler, I. Puzanov, S. S. Agarwala, et al., J. Clin. Oncol. 2015, 33, 2780-2788.
F. Salazar-Onfray, M. Lopez, A. Lundqvist, A. Aguirre, A. Escobar, A. Serrano, C. Korenblit, M. Petersson, V. Chhajlani, O. Larsson, et al., Br. J. Cancer 2002, 87, 414-422.
M. N. López, C. Pereda, M. Ramírez, A. Mendoza-Naranjo, A. Serrano, A. Ferreira, R. Poblete, A. M. Kalergis, R. Kiessling, F. Salazar-Onfray, Investig. Ophthalmol. Vis. Sci. 2007, 48, 1219-1227.
C. Zhang, Z. Zhang, K. S. Lin, J. Lau, J. Zeisler, N. Colpo, D. M. Perrin, F. Benard, Mol. Pharm. 2018, 15, 2116-2122.
C. Zhang, Z. Zhang, H. Merkens, J. Zeisler, N. Colpo, N. Hundal-Jabal, D. M. Perrin, K. S. Lin, F. Benard, Sci. Rep. 2019, 9, DOI 10.1038/s41598-019-50014-5.
C. Zhang, Z. Zhang, J. Zeisler, N. Colpo, K.-S. Lin, F. Benard, ACS Omega 2020, acsomega.0c00310.
P. M. Y. Garcia, G. M. Sigaud, H. Luna, A. C. F. Santos, E. C. Montenegro, M. B. Shah, Phys. Rev. A - At. Mol. Opt. Phys. 2008, 77, 052708.
IAEA, Quality Control in the Production of Radiopharmaceuticals | IAEA TECHDOC-1856, 2018.
Benešová, M.; Schafer, M.; Bauder-Wüst, U.; Afshar-Oromieh, A.; Kratochwil, C.; Mier, W.; Haberkorn, U.; Kopka, K.; Eder, M., Preclinical Evaluation of a Tailor-Made DOTA-Conjugated PSMA Inhibitor with Optimized Linker Moiety for Imaging and Endoradiotherapy of Prostate Cancer. J. Nucl. Med. 2015, 56, 914-920.
Baranski, A.-C.; Schafer, M.; Bauder-Wüst, U.; Wacker, A.; Schmidt, J.; Liolios, C.; Mier, W.; Haberkorn, U.; Eisenhut, M.; Kopka, K.; Eder, M., Improving the Imaging Contrast of ⁶⁸Ga-PSMA-11 by Targeted Linker Design: Charged Spacer Moieties Enhance the Pharmacokinetic Properties. Bioconjugate Chem. 2017, 28, 2485-2492.
Kuo, H.-T.; Pan, J.; Zhang, Z.; Lau, J.; Merkens, H.; Zhang, C.; Colpo, N.; Lin, K.-S.; Benard, F., Effects of linker modification on tumor-to-kidney contrast of ⁶⁸Galabeled PSMA-targeted imaging probes. MolPharm. 2018, 15, 3502-3511.
Benešová, M.; Bauder-Wüst, U.; Schafer, M.; Klika, K. D.; Mier, W.; Haberkorn, U.; Kopka, K.; Eder, M., Linker Modification Strategies To Control the Prostate-Specific Membrane Antigen (PSMA)-Targeting and Pharmacokinetic Properties of DOTA-Conjugated PSMA Inhibitors. J. Med. Chem. 2016, 59, 1761-1775.
Lau, J.; Rousseau, E.; Kwon, D.; Lin, K.; Benard, F.; Chen, X., Insight into the Development of PET Radiopharmaceuticals for Oncology. Cancers 2020, 12, 1312.

1. An in vivo radioisotope targeting construct comprising a biological targeting moiety and a chelator having the structure (I), (II) or (III): 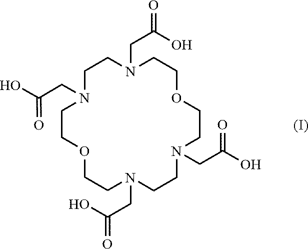 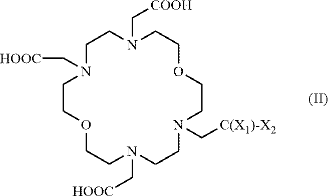 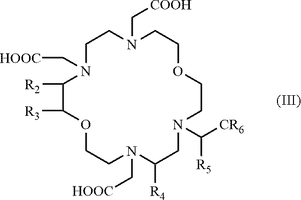
wherein:
X₁ and X₂ are independently O, N or S;
R₂, R₃, R₄, R₅ and R₆ are independently not present or a functional group that can be used to couple the chelator to the biological targeting moiety.

2. An in vivo radioisotope targeting construct as defined in claim 1 comprising the following structure (IV), (V) or (VI): 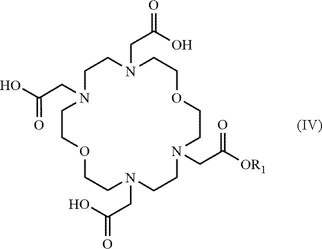 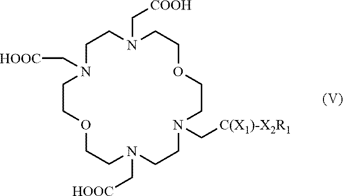 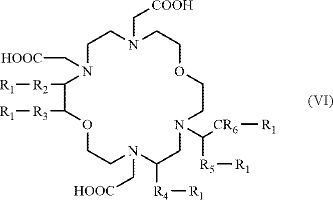
wherein R₁ when present represents the biological targeting moiety.

3. An in vivo radioisotope targeting construct as defined in claim 2 comprising the following structure (VII), (VIII) or (IX): 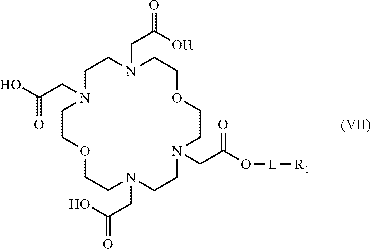 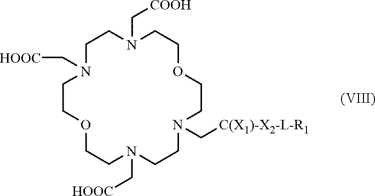 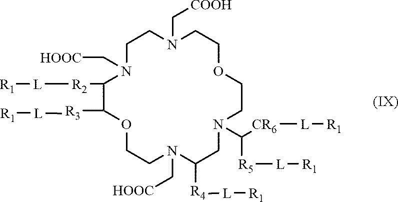
wherein L when present represents a linker.

4. An in vivo radioisotope targeting construct as defined in claim 1, wherein only one of R₂, R₃, R₄, R₅ and R₆ is present.

5. An in vivo radioisotope targeting construct as defined in claim 1, wherein R₂, R₃, R₄, R₅ or R₆ when present are independently a carboxyl, an ester, an amide, an imide, a thioamide, a thioester, a guanidinium, an ether, a thioether, or an amine group.

6. An in vivo radioisotope targeting construct as defined in claim 1, wherein the linker L when present comprises a C₁-C₁₀ hydrocarbon linker that is optionally substituted with one or more heteroatoms or has one or more substituents, an aromatic linker, a cationic linker, an anionic linker, an amino acid linker having between one and ten amino acids, a cyclized amino acid linker, a PEG linker, a cyclized ring linker, an aromatic linker, or a click chemistry linker.

7. An in vivo radioisotope targeting construct as defined in claim 1, further comprising a radiometal chelated by the chelator.

8. An in vivo radioisotope targeting construct as defined in claim 1, wherein the radiometal comprises ²²⁵Ac, ²²⁷Th, ²²⁶Th, ²¹¹At, ⁴⁴Sc, ⁹⁰Y, ⁸⁹Zr,¹⁷⁷Lu, ¹¹¹In, ^{86/89/90}Y, ²¹¹At, ²¹¹Fr, ^{212/2133}Bi ¹⁵³sm, ^{161/166}Ho, ^{165/166}Dy, ^{161/155}Tb, ¹⁴⁰La, ^{142/143/145}Pr, ¹⁵⁹Gd, ^{169/175}Yb, ^{167/170}Tm, ¹⁶⁹Er, ¹⁴⁹Pm, ¹⁵⁰Eu, ⁶⁸Ga, ¹³⁷Cs, or ¹⁴¹Ce.

9. An in vivo radioisotope targeting construct as defined in claim 1, wherein the radiometal comprises ²²⁵Ac, ²¹³Bi, ⁶⁸Ga, ¹⁵⁵Tb, ¹⁷⁷Lu, ¹¹¹In, or ¹³⁷Cs.

10. An in vivo radioisotope targeting construct as defined in claim 1, wherein the radiometal comprises ²²⁵Ac.

11. An in vivo radioisotope targeting construct as defined in claim 1, which has a molar activity of at least 4 MBq/nmol.

12. An in vivo radioisotope targeting construct as defined in claim 1, wherein the targeting moiety comprises a hapten, an antigen, an aptamer, an affibody, an enzyme, a protein, a peptide, an antibody, an antigen-binding fragment of an antibody, a peptidomimetic, a receptor ligand, a steroid, a hormone, a growth factor, a cytokine, a molecule that recognizes cell surface receptors, a lipid, a lipophilic group, or a carbohydrate.

13. An in vivo radioisotope targeting construct as defined in claim 12, wherein the antigen-binding fragment of an antibody comprises an Fab fragment, an F(ab’)₂ fragment, a Fv fragment, an scFv fragment, a minibody, or a diabody.

14. An in vivo radioisotope targeting construct as defined in claim 1, wherein the biological targeting moiety comprises A33 antibody, dihydrotestosterone (DHT), HuMab-5B1, girentuximab, AMG211 bispecific T-cell engager, IAB22M2C minibody, rituximab, obinutuzumab, U36 antibody, plerixafor, pentixafor, NFB, ipilimumab, erlotinib, PD153035, afatinib, cetuximab, panitumumab, ABY-025 affibody, HER2-nanobody, trastuzumab, pertuzumab, GSK2849330, lumretuzumab, 4FMFES, FAPI-04, FAPI-21, FAPI-46, galactose, CB-TE2A-AR06 peptide (with crown substituted for DOTA), BAY 864367 peptide (with crown-bound ligand label instead of 18F labeling), RM2 peptide (with crown substituted for DOTA), SB3 peptide (with crown substituted for DOTA), RM26 peptide, BBN-RGD peptide, Aca-BBN peptide, NeoBOMB1 peptide (with crown substituted for DOTA), exendin-4 peptide, glucose, codrituzumab, EF5, MISO, AZA, HX4, ASTM, LLP2A, peptidomimetic, galacto-RGD peptide, FPP(RGD)2 peptide, RGD-K5 peptide, fluciclatide, alfatide-I, alfatide-II, PRGD2 peptide, αvβ6-BP peptide, CycMSHhex targeting peptides, MMOT0530A antibody, SP peptide, neurotensin, PARPi, a PSMA peptidomimetic, DCFPyL, DCFBC, HuJ591 antibody, durvalumab, nivolumab, pembrolizumab, BMS-986192 adnectin, atezolizumab, MSTP2109A antibody, TATE peptide (octreotate), TOC peptide, NOC peptide, JR11, thymidine, fresolimumab, or bevacizumab.

15. An in vivo radioisotope targeting construct as defined in claim 1, wherein a biological target targeted by the in vivo radioisotope targeting construct comprises: a tumor associated antigen, A33 transmembrane glycoprotein, androgen receptor (AR), CA19.9, carbonic anhydrase 9 (CA-IX), carcinoembryonic antigen, CD8, CD20, CD44v6, C-X-C chemokine receptor type 4 (CXCR4), cytotoxic T-lymphocyte-associated protein 4 (CTLA-4), epidermal growth factor receptor (EGFR), epidermal growth factor receptor 2 (ERBB2), epidermal growth factor receptor 3 (ERBB3), estrogen receptor (ER), fibroblast activation protein a, gastrin-releasing peptide receptor (GRPR), glucagonlike peptide 1 receptor (GLP-1R), glypican 3, integrin α4β1, integrin αvβ3, integrin αvβ6, melanocortin-1 receptor (MC1R), mesothelin, neurokinin1 receptor (NK1R), neurotensin 1 receptor (NTS1R), poly(ADP-ribose) polymerase 1 (PARP1), prostatespecific membrane antigen (PSMA), programmed cell death protein (PD-1), programmed death-ligand 1 (PD-L1), six-transmembrane epithelial antigen of prostate-1 (STEAP1), somatostatin receptor 2 (SSTR2), thymidine kinase, transforming growth factor-beta (TGF-β), or vascular endothelial growth factor receptor (VEGFR).

16. An in vivo radioisotope targeting construct comprising 2,2’,2”,2”’-(1,10-dioxa-4,7,13,16-tetraazacyclooctadecane-4,7,13,16-tetrayl)tetraacetic acid as a chelator.

17. A pharmaceutical composition comprising an in vivo radioisotope targeting construct as defined in claim 1, and a pharmaceutically acceptable carrier, excipient or vehicle.

18. A method of delivering a radioisotope to a selected location within the body of a mammalian subject, the method comprising:
administering an in vivo radioisotope targeting construct as defined in claim 1 bearing the radioisotope to the mammalian subject.

19. A method as defined in claim 18, further comprising allowing the targeting moiety of the in vivo radioisotope targeting construct to enhance the accumulation of the radioisotope at the selected location within the body relative to other locations in the body to selectively deliver radiation to the selected location.

20. A method as defined in claim 18, further comprising a step of forming a chelate comprising the radioisotope and the in vivo radioisotope targeting construct prior to the administering step, wherein the step of forming the chelate construct comprises combining the in vivo radioisotope targeting construct with the radioisotope at a temperature of between about 10° C. and about 65° C. for an incubation period.

21. A method as defined in claim 20, wherein the temperature is between about 15° C. and about 25° C. during the incubation period.

22. A method as defined in claim 20, wherein the incubation period is between about 5 minutes and about 30 minutes.

23. A method as defined in claim 18, wherein the combining step is carried out at a pH in the range of about 5.0 to about 7.4.

24. A method as defined in claim 18, wherein the combining step is carried out in aqueous solution that is substantially free of alcohol.

25. A method as defined in claim 18, further comprising carrying out an imaging procedure to evaluate the localization of the in vivo radioisotope targeting construct within the body, wherein the imaging procedure optionally comprises positron emission tomography (PET) imaging or single-photon emission computerized tomography (SPECT) imaging.

26. A method as defined in claim 18, wherein the in vivo radioisotope targeting construct is used to cause cell death at the selected location within the body by exposing the cells to radiation from the radioisotope.

27. A method as defined in claim 26, wherein the in vivo radioisotope targeting construct is used to cause death of cancer cells at the selected location within the body.

28. A method as defined in claim 26, wherein the radiation comprises alpha radiation.

29. A method as defined in claim 18, wherein the mammalian subject is a human.

30. A metal chelate comprising a metal and a chelator having the following structure (I), (II) or (III): 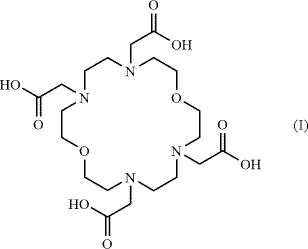 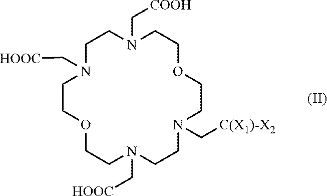 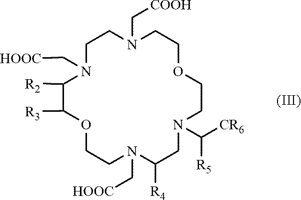
wherein:
X₁ and X₂ are independently O, N or S;
R₂, R₃, R₄, R₅ and R₆ are independently not present or a functional group that can be used to couple the chelator to a biological targeting moiety;
and wherein the metal is selected from the group consisting of: ²²⁵Ac, ²¹³Bi, ⁶⁸Ga, ¹⁵⁵Tb, ¹⁷⁷Lu, ¹¹¹In, or ¹³⁷Cs.

31. The metal chelate of claim 30, wherein the metal is actinium.

32. The metal chelate as defined in claim 30, wherein the metal is actinium-225.

33. The metal chelate as defined in claim 31, wherein the actinium is Ac³⁺.

34. An aqueous solution comprising the metal chelate as defined in claim 30.

35. The aqueous solution as defined in claim 34, wherein the aqueous solution is substantially free of alcohol.

36. A method of forming a metal chelate comprising combining a chelator having the structure (I), (II), (III), (IV), (V), (VI), (VII), (VIII) or (IX) below with a radiometal in an aqueous solution at a temperature of between 15° C. and 25° C. 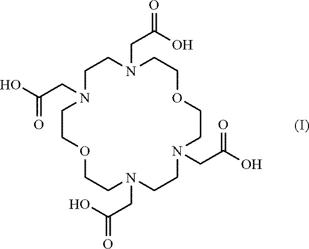 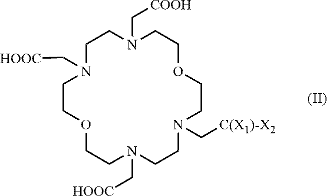 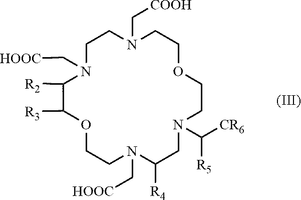 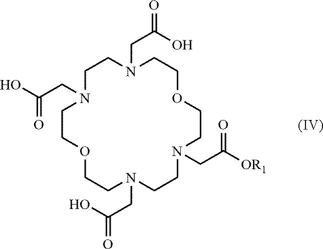 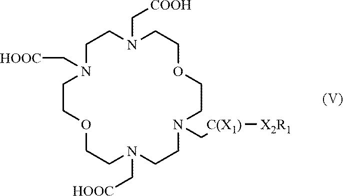 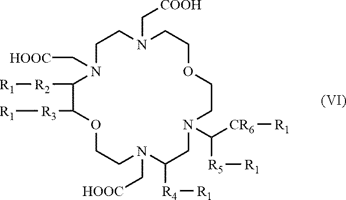 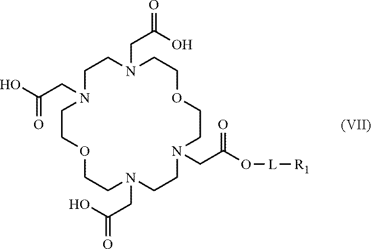 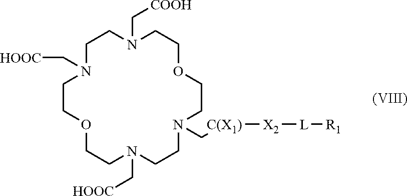 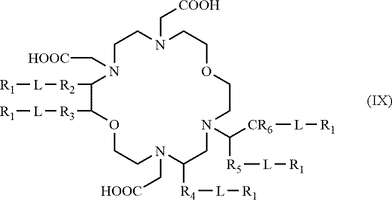
wherein:
X₁ and X₂ are independently O, N or S;
R₂, R₃, R₄, R₅ and R₆ are independently not present or a functional group that can be used to couple the chelator to a biological targeting moiety;
R₁ when present represents a biological targeting moiety; and
L when present represents a linker.

37. The method as defined in claim 36, wherein only one of R₂, R₃, R₄, R₅ and R₆ is present.

38. The method as defined in claim 36, wherein the metal is ²²⁵Ac, ²¹³Bi, ⁶⁸Ga, ¹⁵⁵Tb, ¹⁷⁷Lu, ¹¹¹In, or ¹³⁷Cs.

39. The method as defined in claim 36, wherein the aqueous solution comprises a pH in the range of about 5.0 to about 7.4.

40. A method as defined in claim 36, wherein said combining step is conducted for a period of between about 5 and about 30 minutes.

41. A method as defined in claim 36, wherein the aqueous solution is substantially free of alcohol.

42. A metal chelate as defined in claim 30, that is present in mammalian serum or mammalian blood, optionally human serum or human blood.

43. A metal chelate as defined in claim 30 that is present in a mammal, wherein the mammal is optionally a human.

44. A metal chelate as defined in claim 30 that is present within a mammalian cell, wherein the mammalian cell is optionally a human cell.


SEQUENCE LISTING (7 sequences)

SEQ ID NO: 1 - protein, 8 aa, Artificial Sequence
  synthetic construct
  MOD_RES (1)..(1): D-Phe
  MOD_RES (4)..(4): D-Trp
FCFWKTCT

SEQ ID NO: 2 - protein, 7 aa, Artificial Sequence
  synthetic construct
  MOD_RES (1)..(1): ACETYLATION, Nle
  SITE (2)..(7): cyclized peptide
  MOD_RES (4)..(4): d-Phe
XDHFRWK

SEQ ID NO: 3 - protein, 2 aa, Artificial Sequence
  synthetic construct
GG

SEQ ID NO: 4 - protein, 3 aa, Artificial Sequence
  synthetic construct
EEE

SEQ ID NO: 5 - protein, 4 aa, Artificial Sequence
  synthetic construct
GSGS

SEQ ID NO: 6 - protein, 2 aa, Artificial Sequence
  synthetic construct
DD

SEQ ID NO: 7 - protein, 2 aa, Artificial Sequence
  Synthetic construct
EE